(19) United States
(12) Patent Application Publication
(10) Pub. No.: US 2020/0383964 A1
(43) Pub. Date: Dec. 10, 2020

(54) MITOCHONDRIA TARGETING, MPTP REGULATION, AND THE REDUCTION OF MITOCHONDRIAL REACTIVE SPECIES

(71) Applicant: Christopher Duke, San Diego, CA (US)

(72) Inventor: Christopher Duke, San Diego, CA (US)

(21) Appl. No.: 16/770,553

(22) PCT Filed: Dec. 5, 2018
(86) PCT No.: PCT/US2018/063981

§ 371 Date: Jun. 5, 2020

Related U.S. Application Data
(60) Provisional application No. 62/595,736, filed on Dec. 7, 2017.
(60) Provisional application No. 62/651,117, filed on Mar. 31, 2018.
(60) Provisional application No. 62/728,838, filed on Sep. 9, 2018.

(51) Int. Cl. A61K 31/4433; A61K 9/127; A61K 31/4422

(52) U.S. Cl. CPC A61K 31/4433; A61K 31/4422; A61K 9/127

(57) ABSTRACT

Provided herewith is a compound that reacts with a reactive lipid species to form a non-toxic product or an active product. Also provided is a method of reducing a reactive lipid species. Additionally provided is a method of treating a mitochondrion in a patient comprising a mitochondrial dysfunction. In other embodiments, a compound is provided that reacts with an activating agent present at a mitochondrion to form an active product that inhibits or enhances the opening of a mitochondrial permeability transition pore (MPTP). In additional embodiments, a method of inhibiting or enhancing the opening of an MPTP is provided. Further provided is a nonpolar compound capable of crossing the blood-brain barrier (BBB) into the central nervous system (CNS).

### CROSS-REFERENCE TO RELATED APPLICATIONS

This application claims the benefit of U.S. Provisional Application No. 62/595,736, filed Dec. 7, 2017; U.S. Provisional Application No. 62/651,117, filed Mar. 31, 2018; and U.S. Provisional Application No. 62/728,838, filed Sep. 9, 2018, all three incorporated by reference herein in their entirety.

### BACKGROUND OF THE INVENTION

### (1) Field of the Invention

The present application generally relates to the targeting of mitochondria, the regulation of the mitochondrial permeability transition pore (MPTP), and the reduction of mitochondrial reactive species and/or mitochondrial reactive lipid species. More specifically, the application is directed to compounds and methods for targeting the mitochondria, reducing mitochondrial reactive species and/or mitochondrial reactive lipid species, inhibiting or enhancing the opening of the MPTP using prodrugs and/or inactive compounds, and treating diseases having a mitochondrial component with those prodrugs and/or inactive compounds.

### (2) Description of the Related Art

Mitochondria play a critical role in the health and normal functioning of a cell. It is the main energy generator for the cell, the major cellular reactive oxygen species (ROS) producer, and can release pro-apoptotic proteins into the cytoplasm to initiate cell death. Slight alterations or increased permeability of the mitochondria (mitochondrial dysfunction) can create drastic changes to a cell's health. Because of their importance to cellular health, mitochondria are central factors to many diseases, including those that involve oxidative stress, inflammation, and mitochondrial dysfunction. The opposite occurs in cancer where there is a decrease in mitochondrial permeability, thus making treatment more difficult.

Two of the most important factors responsible for the onset and progression of mitochondrial dysfunction are the opening of the mitochondrial permeability transition pore (MPTP), and the increased generation of reactive species and reactive lipid species. Both of these factors are heavily intertwined, where the onset of one usually triggers the onset of the other, in a deleterious cycle that affects the health of both the mitochondria and the cell.

The opening of the mitochondrial permeability transition pore causes increased mitochondrial reactive species and reactive lipid species levels, large mitochondrial matrix Ca⁺² fluctations, mitochondrial swelling, a decrease in cellular energy production, mitochondrial membrane depolarization, the release of pro-apoptotic factors into the cytoplasm, and eventual cellular death (Rao et al., 2014; Hurst et al., 2017). For diseases and conditions characterized by increased permeability of the mitochondria due to excessive MPTP opening, treatments targeting the MPTP and inhibiting its opening in both cellular and animal models have been effective, although new mechanisms and approaches to inhibit MPTP are desperately needed to treat humans.

Inhibiting the MPTP can therefore be an effective treatment for diseases involving mitochondrial dysfunction. Several compounds can affect MPTP opening. Glyoxal derivatives of α-oxoaldehydes and α-oxoketones specifically target the MPTP along the inner mitochondrial membrane (IMM), and bind to an arginine residue on the MPTP, located on the matrix side of the IMM. The binding is either covalent or non-covalent, and provided an either irreversible or reversible effect, depending on the molecule that was used. Also, no other noticeable effects were displayed by the isolated mitochondria after treatment, which reflected that the molecules specifically targeted the critical arginine residue located on the MPTP, and did not affect cellular respiration or the functioning of other gates and channels, thus showing a high degree of specificity toward MPTP arginine residue(s) (Eriksson et al., 1998; Speer et al., 2003; Johans et al., 2005; Linder et al., 2001; Sileikyte et al., 2015).

Some of the glyoxal compounds, such as phenylglyoxal, irreversibly bind to the MPTP under physiological mitochondrial matrix conditions. This indicates that this technique can provide long-term beneficial health effects. Phenylglyoxal is also highly specific towards binding with arginine, and therefore it displays a very controlled reaction (Takahashi 1977). This is in contrast to typical uncontrolled lipid peroxidation decomposition byproducts that undergo uncontrolled reactions with various amino acids in the mitochondria and elsewhere in the body, to produce both reversible and irreversible reactions with unknown targets, unknown consequences, and unknown side effects. Some of the implications from these random lipid decomposition byproduct reactions that have already been proven include the inhibition of proteins on the electron transport chain, or even the induction of the MPTP itself, even at femtomolar concentrations, as discussed in Anderson et al., 2012 and Kristal et al., 1996. As a result, by targeting only one amino acid, with a high degree of specificity, while exhibiting little to no toxicity—as demonstrated when these glyoxal-derivative compounds were incubated at high concentrations with isolated mitochondria—we can help protect the cell from further damage in a very controlled and safe mechanism.

Just as it is possible to selectively target this arginine residue on the MPTP and close it with nonpolar phenylglyoxal derivatives, the MPTP can also be activated with polar phenylglyoxal derivative activating agents, also described in Johans et al., 2005. If this pore is selectively targeted and opened, then you can help kill cancer cells as Johans demonstrated, by inducing increased mitochondrial matrix ROS levels, drastic mitochondrial matrix Ca⁺² fluctuations, mitochondrial swelling, a decrease in cellular energy production, mitochondrial membrane depolarization, and the release of pro-apoptotic factors into the cytoplasm to cause necrosis and/or apoptotic cellular death. Compounds that promote the opening of the MPTP are further described in Johans et al, 2005; Bhutia et al., 2016; and Sun et al., 2014.

The problem with the glyoxyl derivatives that are effective in opening or closing the MPTP are that they are reactive, as a result of having a strong electron-withdrawing group on the adjacent atom, a characteristic of glyoxyls and other α-dicarbonyl compounds. Therefore, if these molecules were administered by themselves into the body, they would react very quickly with any surrounding tissue, and any arginine residues they encounter, and are thus ineffective unless they are delivered or produced at or near the mitochondrial matrix. Therefore, to use this type of MPTP regulating technology, a highly specific drug delivery system is advantageous.

The second major factor responsible for the onset and progression of mitochondrial dysfunction, as previously mentioned, is the excessive generation of reactive species and reactive lipid species in the mitochondria. Normally, reactive species, including reactive oxygen species, are present at low levels, and are used as cellular signaling molecules, which are vital for the typical day-to-day tasks of the cell. However, if a cell undergoes an injury or is associated with a particular disease, they might overproduce these reactive species, causing the cell's standard antioxidant capacity to be overwhelmed, resulting in harm and a further escalation of damage to the cell. For example, these reactive species and reactive lipid species can cause DNA and mtDNA mutations, alter protein expression levels, inhibit vital cellular pathways including energy production pathways, and even activate new pathways including pro-apoptotic pathways that eventually lead to cellular death (Guo et al., 2014). As a result, in order to help treat diseases that are characterized by mitochondrial dysfunction, oxidative stress and inflammation, it is important to target and reduce these intramitochondrial levels of reactive species and reactive lipid species.

It is important to note that reactive species and reactive lipid species are heavily intertwined with the MPTP, and the onset of one can cause the onset of the other. For example, reactive species directly and indirectly react with the MPTP and induce its opening (Zhang et al., 2016); reactive lipid species and peroxidized cardiolipin, including hydroperoxide cardiolipin, interact with the adenine nucleotide translocator (ANT) and inhibit it, thus inducing MPTP opening (Nakagawa 2004); reactive lipid species and peroxidized cardiolipin, including hydroperoxide cardiolipin, have been shown to interact differently than their non-peroxidized lipid counterparts with various proteins along the IMM, including those that take part in energy production, which results in the decrease of ATP generation while increasing reactive species production—and as already discussed, these reactive species can directly interact with MPTP and induce its opening (Petrosillo et al., 2008); and finally, reactive lipid species and peroxidized cardiolipin, including peroxyl radical cardiolipin, undergo decomposition reactions that create highly reactive lipid peroxidation byproduct molecules that have been shown to bind directly to the MPTP and induce its opening (Anderson et al., 2012; Kristal et al., 1996). Similarly, the induction of the MPTP will lead to the influx of more reactive species, which could harm the cell and damage components of the electron transport chain, thus causing the creation of even more reactive species (Zorov et al., 2014).

There is a need for additional compounds and methods that affect mitochondrial dysfunction, including reducing reactive species and reactive lipid species, and controlling the MPTP. The present invention provides those compounds and methods. In many cases, the basic chemical structures and mechanisms described in WO 2016/023015, WO 2017/049305 and WO 2018/102463 are utilized, and are incorporated by reference herewith.

### BRIEF SUMMARY OF THE INVENTION

Provided herewith is a compound that reacts with a reactive lipid species to form a non-toxic product or an active product, wherein the compound comprises any of the following structures:

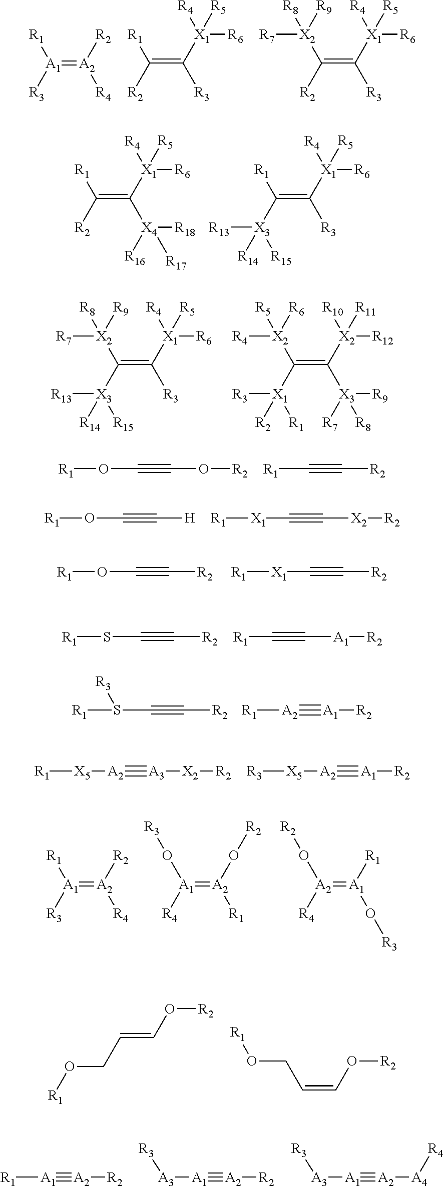

wherein

A₁, A₂, A₃ and A₄ are each independently C, S, N, Si or P;

X₁, X₂, X₃ and X₄ are each independently, O, N, S, or P; and

R₁-R₁₈ are each independently alone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., N, P, or), electron-withdrawing group, a halogen, a resonating or non-resonating moiety, a conjugated or non-conjugated moiety, substituted or unsubstituted arylalkyl, or substituted or unsubstituted heteroarylalkyl, wherein where more than one R groups are present, two R groups can join together to form a ring.

Also provided is a method of reducing a reactive lipid species. The method comprises contacting the reactive lipid species with any of the above compounds.

Additionally provided is a method of treating a mitochondrion in a patient comprising a mitochondrial dysfunction. The method comprises contacting the mitochondrion with any of the above compounds.

In other embodiments, a compound is provided that reacts with an activating agent present at a mitochondrion to form an active product that inhibits or enhances the opening of a mitochondrial permeability transition pore (MPTP).

In additional embodiments, a method of inhibiting or enhancing the opening of an MPTP is provided. The method comprises contacting a mitochondrion with any of the compounds immediately above, in a manner sufficient for the compound to react with the activating agent to form the active product.

Further provided is a nonpolar compound capable of crossing the blood-brain barrier (BBB) into the central nervous system (CNS). In these embodiments, the compound reacts with an activating agent in the CNS to form an active cationic product that reacts with a mitochondrial reactive lipid species and/or inhibits or enhances the opening of a mitochondrial permeability transition pore (MPTP).

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

As used herein, “a free radical, an ROS [reactive oxygen species] or another reactive species” includes any of the following: organic peroxides, peracids, dioxygenyls, hypochlorite, reactive halogenated compounds, peroxy salts, alkoxides, reactive phosphorous oxides, peroxynitrite, nitric acid, sulfuric acid, phosphoric acid, nitrosoperoxycarbonate, carbonate radical, dinitrogen trioxide, nitrogen dioxide, hydroxyl ion, nitrous oxide, peroxynitrate, peroxynitrous acid, nitroxyl anion, nitrous acid, nitryl chloride, nitrosyl cation, hypochloric acid, hydrochloric acid, lipid peroxyl, peroxyl, peroxynitrite, alkyl peroxides, alkyl peroxynitrites, perhydroxyl radicals, diatomic oxygen, free electrons, sulfur dioxide, free radicals, superoxide, hydrogen peroxide, hydroxyl radical, nitric oxide, peroxynitrite, hypochlorous acid, persulfides, polysulfides, thiosulfates, organic radicals, peroxy radical, alkoxy radical, thiyl radical, sulfonyl radical, thiyl peroxyl radical, sulfur polycations, sulfides, oxoacids, oxoanions, sulfur trioxide, sulfites, pyrosulfuric acid, sodium dithionite, dithionite, oxyhalides, sulfuric acid derivatives, hydrogen sulfide, sulfurous acid, reactive oxygen species, reactive nitrogen species, reactive sulfur species, reactive phosphorous species, singlet oxygen, dioxygen, triplet oxygen, ozone (including atmospheric ozone), reactive nitrogen oxides, reactive sulfur oxides, ozonide, dioxygenyl cation, atomic oxygen, carbon monoxide, peroxides, organic hydroperoxides, nitrosoperoxycarbonate anion, nitrocarbonate anion, dinitrogen dioxide, nitronium, atomic oxygen, hydroxyl anion, lipid radicals, cardiolipin radicals, lipid peroxyl radicals, lipid alkoxyl radicals, cardiolipin peroxyl radicals, cardiolipin alkoxyl radicals, fatty acid radicals, fatty acid alkoxyl radicals, fatty acid peroxyl radicals, lipid hydroperoxides, cardiolipin hydroperoxides, fatty acid hydroperoxides, lipid-lipid peroxides, lipid peroxides, cardiolipin peroxides, cardiolipin-lipid peroxides, and cardiolipin-cardiolipin peroxides.

As used herein, “a reactive lipid species or a mitochondrial reactive lipid species” includes any of the following: lipid radicals, cardiolipin radicals, lipid peroxyl radicals, lipid alkoxyl radicals, cardiolipin peroxyl radicals, cardiolipin alkoxyl radicals, fatty acid radicals, fatty acid alkoxyl radicals, fatty acid peroxyl radicals, lipid hydroperoxides, cardiolipin hydroperoxides, fatty acid hydroperoxides, lipid-lipid peroxides, lipid peroxides, cardiolipin peroxides, cardiolipin-lipid peroxides, and cardiolipin-cardiolipin peroxides.

Non-limiting examples of free radicals are superoxide, hydrogen peroxide, hydroxyl radical, perhydroxyl radical, nitric oxide, peroxynitrite, peroxyl radicals, organic radicals, peroxy radical, lipid radicals, cardiolipin alkoxyl radicals, cardiolipin peroxyl radicals, fatty acid peroxyl radicals, cardiolipin radicals, alkoxy radical, thiyl radical, sulfonyl radical, and thiyl peroxyl radical; Nonlimiting examples of ROS are singlet oxygen, dioxygen, triplet oxygen, ozone (including atmospheric ozone), nitrogen oxides, ozonide, dioxygenyl cation, atomic oxygen, sulfur oxides, ammonia, hydroxyl anion, carbon monoxide, lipid radicals, cardiolipin radicals, lipid alkoxyl radicals, cardiolipin alkoxyl radicals, fatty acid radicals, fatty acid alkoxyl radicals, cardiolipin radicals, peroxides including (but not limited to) hydrogen peroxide, alkyl peroxides, peroxyl radicals, organic hydroperoxides, peroxide ion, organic peroxides, peracids, peroxysulfuric acid, peroxymonosulfur acid, peroxydisulfuric acid, peroxyphosphoric acid, meta-chloroperoxybenzoic acid, peresters, peracetic acid, peroxy anion, performic acid, nitrosoperoxycarbonate anion; nitrocarbonate anion, dinitrogen dioxide, nitronium, atomic oxygen, lipid peroxyl radicals, cardiolipin peroxyl radicals, fatty acid peroxyl radicals, lipid hydroperoxides, cardiolipin hydroperoxides, fatty acid hydroperoxides, lipid-lipid peroxides, lipid peroxides, cardiolipin peroxides, cardiolipin-lipid peroxides, and cardiolipin-cardiolipin peroxides.

In all compounds or products provided herein, where relevant and not otherwise specified or obvious, X, Y, Z or n is an integer from 1 to 1,000,000 and any R group is a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., O, N, P, or S), a halogen, substituted or unsubstituted arylalkyl, or substituted or unsubstituted heteroarylalkyl. Where more than one R groups are present, two R groups can join together to form a ring structure. These R groups can be conjugated or unconjugated groups, electron donating and/or electron withdrawing conjugated and/or unconjugated groups, resonating or non-resonating groups, ionic or non-ionic, cationic, anionic, or neutral, polar or non-polar, bulky or non-bulky, or lipophilic or non-lipophilic.

Any R group can be duplicated any number of times in a series or at different R group positions. A particularly common repeated moiety in the compounds provided herein is a vinyl ether structure.

These R groups can be conjugated or unconjugated groups, electron donating and/or electron withdrawing conjugated and/or unconjugated groups, reactive species and/or reactive lipid species sensitive, resonating or non-resonating groups, cis or trans, ionic or non-ionic, charged or uncharged, cationic, anionic, polar or non-polar, aromatic, non-aromatic, or anti-aromatic, bulky or non-bulky, and lipophilic or non-lipophilic.

The skilled artisan would also understand that any of the reactive moieties reactive with free radicals, ROS or other reactive species, either as a monomer or using any of the polymer backbones, described in PCT Patent Application Publications WO 2016/023015, WO 2017/049305 and WO 2018/102463 can be modified to produce these mitochondrial targeting prodrugs and prodrugs targeting other subcellular organelles or structures. Additionally, unless otherwise indicated, any non-toxic or active product released, by reaction with a reactive species or enzyme, from a compound described herein can be any reaction product described in WO 2016/023015, WO 2017/049305 or WO 2018/102463.

The compounds provided in this specification can be monomeric, generally having a molecular weight less than 1000, or oligomeric or polymeric, generally having a molecular weight more than 1000. When the prodrug is a polymer, any polymeric backbone, including but not limited to any of the polymeric backbones provided in WO 2016/023015, WO 2017/049305 or WO 2018/102463, or any portion thereof, can be utilized as appropriate.

The present invention is based in part on the indication that mitochondrial reactive species and reactive lipid species can both directly and indirectly affect the induction of the MPTP, while the MPTP can also directly and indirectly affect the levels of reactive species and reactive lipid species as well. Because of this strong linkage between the two entities, targeting and treating one of these groups, whether the MPTP or reactive species and/or reactive lipid species, will often help treat the other, whether directly or indirectly.

The present invention thus provides, in part, prodrugs and/or inactive compounds that target and react with reactive species and/or reactive lipid species present at a mitochondrion to form an activated product, e.g., a drug, that directly inhibits or enhances the opening of a mitochondrial permeability transition pore (MPTP); and prodrugs and/or inactive compounds that target and react with an reactive species and/or reactive lipid species present at a mitochondrion that indirectly affect the opening or closing of the MPTP.

### Reduction of Reactive Species

In some embodiments, a compound that reacts with a reactive lipid species to form a non-toxic product or an active product is provided. The compound in these embodiments comprises any of the following structures:

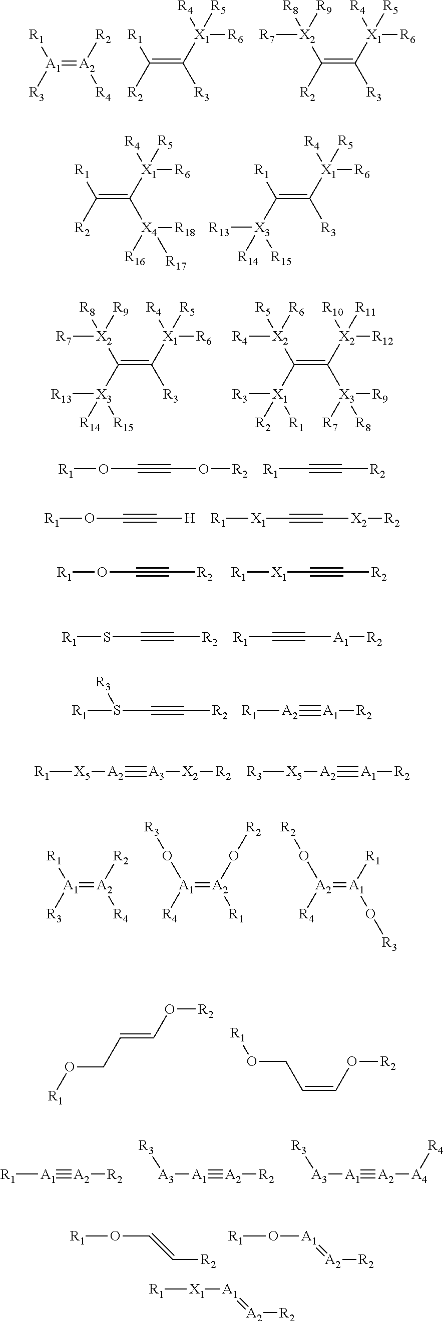

wherein

A₁, A₂, A₃ and A₄ are each independently C, S, N, Si or P;

X₁, X₂, X₃ and X₄ are each independently O, N, S, or P; and

R₁-R₁₈ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., O, N, P, or S), electron-withdrawing group, a halogen, a resonating or non-resonating moiety, a conjugated or non-conjugated moiety, substituted or unsubstituted arylalkyl, or substituted or unsubstituted heteroarylalkyl, wherein where more than one R groups are present, two R groups can join together to form a ring.

In some embodiments, the compound comprises a dienol ether, a divinyl thioether, a dienamine, an enol ether, a vinyl thioether, an enamine, or a combination thereof. Such compounds are generally provided in WO 2018/102463, but the compounds of the present invention are designed to react with reactive lipid species, such as those found in the inner mitochondrial membrane. However, these embodiments are not limited to compounds that react with reactive lipid species at the mitochondria, but also encompass reaction with reactive lipid species in any tissue, cell, organelle, or outside of a living system.

The use of an enol ether, vinyl thioether, enamine, dienol ether, divinyl thioether, and/or dienamine provides the advantage that the alkene reacts with all reactive species and produces specific functional groups upon reaction. Also, the vinyl ether group is one of the first targets for reactive species reactions, compared to traditional alkenes (Stadelmann-Ingrand et al., 2001). The benefit of a dienol ether, dienamine, or divinyl thioether, and/or combinations, instead of just a single electron donating group adjacent to the alkene, is that the extra electron donating group increases the frequency in which the alkene receives electron density, thus forming a formal negative charge on one of the carbons in the alkene and allowing for faster reactions with reactive species and free radicals. This allows for not only stable prodrugs (since the enol ether functional group is already highly present throughout the mammalian body), but also allows for very sensitive prodrugs to activate upon the presence of reactive species and free radicals, which is a common component of many pathological diseases. Also, by using two electron-donating groups on either side of the alkene, dicarbonyl groups (see below) that cannot be made physiologically in the mitochondrial matrix any other way.

In some embodiments, the compound forms an active product upon reaction with the reactive lipid species. Examples of such active products are a specific binding agents, biocides, fragrances, cosmetics, dyes, antioxidants, fertilizers, nutrients, metabolites, food ingredients or pharmaceuticals, as they are described in WO 2017/049305 and WO 2018/102463, incorporated by reference.

In various embodiments, upon reaction with the compound, the reactive species or reactive lipid species (—O—O—) radical or non-radical moiety will be reduced to a (—O—) radical or non-radical moiety. An example of this mechanism is provided. Important reactive lipid species that are reduced upon reaction with the invention compounds include cardiolipin hydroperoxides and cardiolipin peroxyl radicals. The reduction of these cardiolipin reactive species helps restore normal functioning to the mitochondria.

It is noted that the compounds of the present invention that are intended to target the mitochondria are cations, which are directed to the negatively charged mitochondria.

In some embodiments, the non-toxic or active product comprises an α-hydroxy aldehyde, for example glyceraldehyde, upon reaction of the compound with a reactive species, e.g., a reactive lipid species. Nonlimiting examples of such compounds include the following:

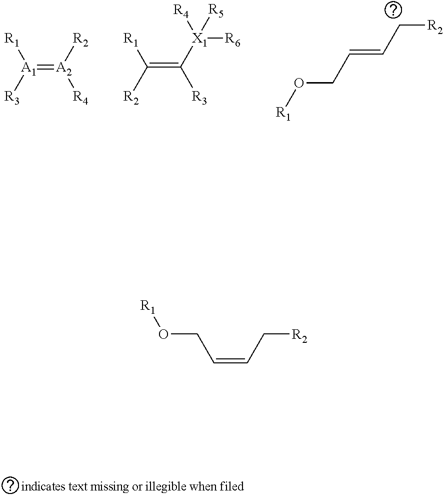

wherein

A1 and A2 are independently C, S, N, Si or P; and

X₁ is O, N, S, or P; and

R1, R2, R3, R4, R5, and R6 are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., O, N, P, or S), electron-withdrawing group, a halogen, a resonating or non-resonating moiety, a conjugated or non-conjugated moiety, substituted or unsubstituted arylalkyl, or substituted or unsubstituted heteroarylalkyl. Where more than one R groups are present, two R groups can join together to form a ring structure.

In some of these embodiments, the α-hydroxy aldehyde is a sugar. Nonlimiting examples of sugars that can be made in these embodiments include trioses, tetroses, ketoses and aldoses.

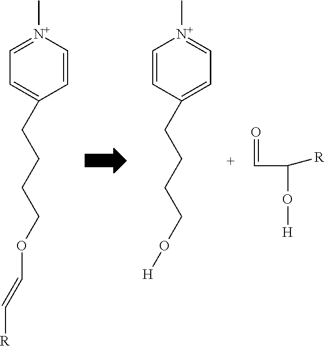

The mechanism above shows an example of a compound targeting mitochondrial lipid reactive species. The cationic charge targets the compound to the mitochondria. The newly formed hydroxyl group on the cationic product is quickly removed from the lipid bilayer. That product does not target the MPTP.

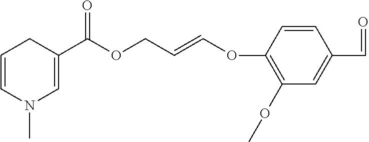 [SMILES: COc1cc(C=O)ccc1O/C=C/COC(=O)C1=CN(C)C=CC1]

The above compound will create vanillin and glyceraldehyde upon reaction with a reactive species and will not target the MPTP.

In some of these embodiments, the reactive lipid species is a cardiolipin hydroperoxide or a cardiolipin peroxyl radical.

The above illustration shows a general mechanism of when polyunsaturated fatty acids, such as cardiolipin, are oxidized by free radical reactions, and form either a peroxyl or hydroperoxide fatty acid. Both of these are very toxic to the cell, specifically with cardiolipin. Also the hydroperoxide fatty acid is very stable, so a large accumulation of these can occur in an inner mitochondrial membrane. Removing these reactive peroxyl or hydroperoxide groups from these fatty acids allow the mitochondria to regain normal functioning by creating the corresponding hydroxyl fatty acids. Vinyl ethers remove fatty acid peroxyl radicals very quickly. Peroxyl radicals are reactive, while the hydroperoxide is stable and not that reactive. Fortunately, there is going to be an interconversion of hydroperoxide cardiolipin and peroxyl radical cardiolipin since they hydrogen is easily abstracted by perhydroxyl radicals which are formed from superoxide. Without being bound by any particular mechanism, it is believed that this is how fatty acid decomposition and initiation of these free radical chain reactions in lipid bilayers occurs, by the perhydroxyl radical entering the lipid bilayer, abstracting a hydrogen from hydroperoxide lipids, which then causes the free radical chain reaction and lipid peroxidation chemical reactions. Although the reactions utilized herein do not recreate the unreacted cardiolipin molecule, they do create the alcohol version that behaves identical to the original unreacted cardiolipin molecule. Since vinyl ethers react specifically with peroxyl radical lipids, and turn those into alkoxyl radicals, which will very

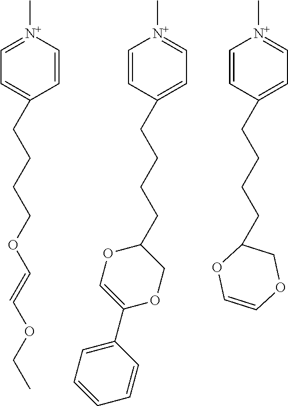 [SMILES: CCO/C=C/OCCCCc1cc[n+](C)cc1 ; C[n+]1ccc(CCCCC2COC(c3ccccc3)=CO2)cc1 ; C[n+]1ccc(CCCCC2COC=CO2)cc1]

quickly form an alcohol group, we can both remove the peroxyl radical lipids, as well as the hydroperoxide cardiolipin molecules—as already mentioned they keep reforming to the peroxyl radical lipid. Therefore, this process converts these harmful lipids into benign lipids by creating the alcohols from them. Since plasmalogens (vinyl ethers) terminate free radical chain reactions, this process also terminate these chain reactions. As shown in the above illustration, the compound, upon reaction with the peroxy radicals, create an active or non-toxic product that, depending on its structure, can either target the MPTP, or not.

The above compounds have permanent cationic charges and are designed for rapid insertion into the inner mitochondrial membrane (IMM). Also, these compounds have a cationic group at the far end of its aromatic moiety from the lipophilic chain. This allows the compound to insert itself as far as possible into the IMM, and potentially below the negative charge on the phospholipid bilayer. This is apparently what happens with the fluorescent molecule APP+ which is a small lipophilic cationic compound, and upon depolarization of the mitochondria, it stays attached to the membrane, which suggests that it is being blocked potentially by some parts of the phospholipids in the bilayer. A likely explanation of this is that the cationic group is somehow trapped partially, and potentially positioned below some parts of the lipids, which does not allow them to leave very well, especially since there is a small lipophilic chain associated with the molecule.

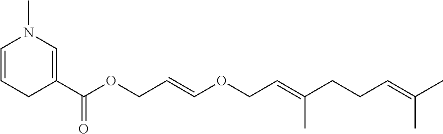 [SMILES: CC(C)=CCC/C(C)=C/CO/C=C/COC(=O)C1=CN(C)C=CC1]

The above compound targets cardiolipin hydroperoxides, crosses the blood brain barrier, and is activated by enzymes to form the following positive charged molecule once in the tissues. These advantages are further discussed in detail below.

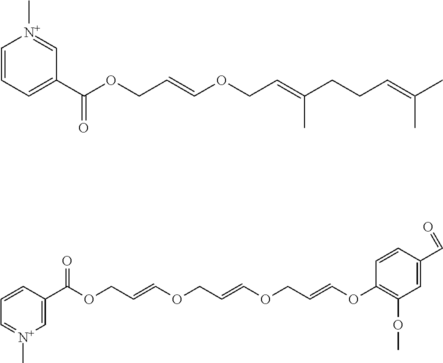 [SMILES: CC(C)=CCC/C(C)=C/CO/C=C/COC(=O)c1ccc[n+](C)c1 ; COc1cc(C=O)ccc1O/C=C/CO/C=C/CO/C=C/COC(=O)c1ccc[n+](C)c1]

The above compound will produce three glyceraldehyde molecules and vanillin upon reaction with a reactive species. Because of its long lipophilic chain, it primarily targets reactive lipid species, including cardiolipin peroxide species. It does not target MPTP since no alpha-dicarbonyl molecules are made here.

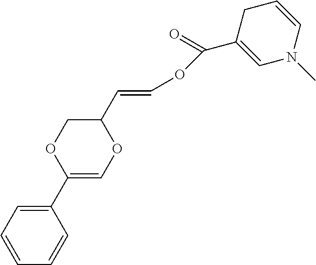 [SMILES: CN1C=CCC(C(=O)O/C=C/C2COC(c3ccccc3)=CO2)=C1]

The above compound creates a tetrose and the MPTP targeting phenylglyoxal product upon reaction with a reactive species.

These reactive species-reacting compounds can also be alkynes, similar to the alkynes described in WO 2018/102463, incorporated by reference. Examples of such compounds are the following

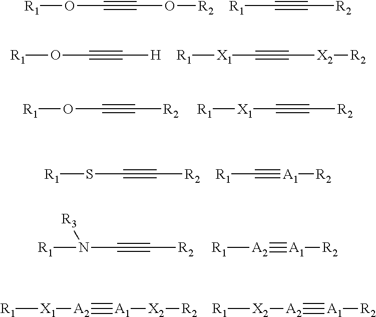

Without being bound to any particular reaction mechanism, the above illustration illustrates a general alkyne reaction mechanism with reactive species. An alkynic ether reacting with a reactive species by free radical addition to form a hydroxyl group and a radical is shown. The radical quickly binds to oxygen, and this undergoes a hemiacetal reorganization to form a dicarbonyl group with an ester. This can undergo further decomposition by esterase or chemically to produce a carboxylic acid (here, pyruvic acid) and an alcohol. Alkynic ethers are useful compounds in these embodiments because they are generally reactive with free radicals since they have electron rich pi bonds.

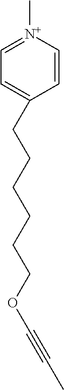 [SMILES: CC#COCCCCCCc1cc[n+](C)cc1]

The above compound is another example of a useful alkyne. Having a cationic charge, it targets the mitochondria.

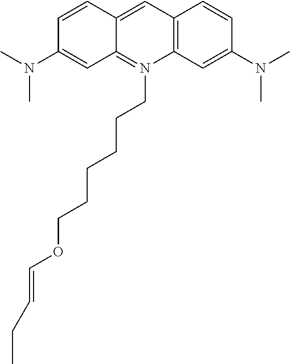

The above compound is a derivative of nonyl acridine orange. Nonyl acridine orange targets cardiolipin, especially in the inner mitochondrial membrane, and is used as a specific labeling agent for cardiolipin. As such, the above compound utilizes its non-polar chain in the IMM lipid bilayer, and the vinyl ether group targets the peroxidized cardiolipin hydroperoxides and peroxyl radicals.

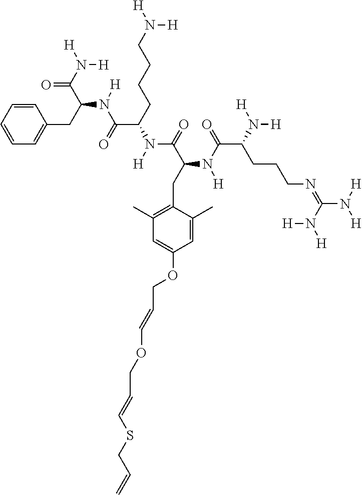 [SMILES: C=CCS/C=C/CO/C=C/COc1cc(C)c(C[C@H](NC(=O)[C@H](N)CCCN=C(N)N)C(=O)N[C@@H](CCCCN)C(=O)N[C@@H](Cc2ccccc2)C(N)=O)c(C)c1]

The above compound is a derivative of an SS peptide, specifically SS-31. SS-31 targets cardiolipin molecules, specifically, peroxidized cardiolipin molecules. SS-31 and other SS peptides help to prevent the peroxidase activity of complex III and promote the oxidase activity. By adding vinyl ether groups onto the tryptophan amino acid, or other amino acids or derivatives, the chain is designed to insert into the bilayer, and help reduce the peroxides and peroxyl radicals of cardiolipin. This compound thus provides more than one function, including stabilizing the cardiolipin—protein complex oxidase activity, and reducing the peroxidized cardiolipin chains. This will provide specificity and allows targeting of the mitochondria without the use of cations. Also, the thiol allyl group will form allyl mercaptan upon reactive species and reactive lipid species oxidation. Allyl mercaptan is one of the most potent HDAC inhibitor, which is an example of another therapeutic molecule or medication we can create in the process that can help treat multiple pathways.

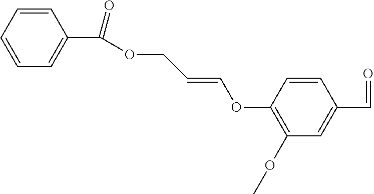 [SMILES: COc1cc(C=O)ccc1O/C=C/COC(=O)c1ccccc1]

The compound above targets all reactive species, including lipid reactive species. Since this compound does not have a cationic charge on it, it will cross the blood-brain barrier, and target reactive species everywhere, not just the mitochondria. Also, it will form vanillin, benzilic acid, and glyceraldehyde as its metabolic byproducts.

While many compounds provided herewith have a cationic charge to target the mitochondria, also provided are similar compounds, but without a cationic charge. Those neutrally charged compounds are not directed to the mitochondria, but are useful to neutralize reactive species throughout the body, and would be able to cross the blood-brain barrier. Examples of these are below. The compound immediately below produces phenylglyoxal upon reaction with a reactive species, which will target and inhibit the MPTP; the other compound does not produce an MPTP-reactive compound, but is only designed to reduce reactive species like hydroperoxide lipids.

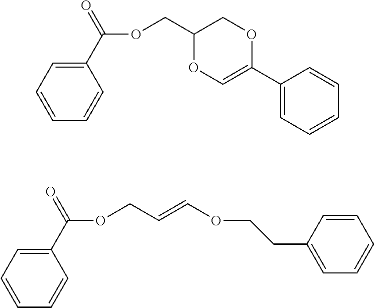 [SMILES: O=C(OCC1COC(c2ccccc2)=CO1)c1ccccc1 ; O=C(OC/C=C/OCCc1ccccc1)c1ccccc1]

In other embodiments, compounds that generate an α-dicarbonyl upon reaction with a reactive species or a reactive lipid species are provided. The compounds comprise the following structures

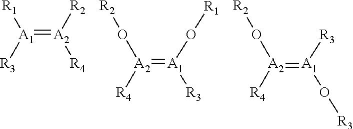

wherein

A1 and A2 are independently C, S, N, Si or P; and

R1, R2, R3 and R4 are each a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., O, N, P, or S), electron-withdrawing group, a halogen, a resonating or non-resonating moiety, a conjugated or non-conjugated moiety, substituted or unsubstituted arylalkyl, or substituted or unsubstituted heteroarylalkyl. Where more than one R groups are present, two R groups can join together to form a ring structure.

Since many α-dicarbonyl compounds bind to arginine residues on the MPTP and either inhibit or enhance MPTP opening, these compounds are particularly useful for affecting MPTP opening. See discussion below.

Also provided herewith is a method of reducing a reactive lipid species. The method comprises contacting the reactive lipid species with any of the compounds described above.

In some embodiments of these methods, the compound reduces cardiolipin hydroperoxide or cardiolipin peroxyl radical to a (—O—) radical or non-radical moiety. In other embodiments, the active product inhibits or enhances the opening of a mitochondrial permeability transition pore (MPTP). In some of those embodiments, the non-toxic or active product comprises an α-dicarbonyl.

### Targeting the Mitochondrial Permeability Transition Pore (MPTP)

Also provided herewith are compounds that react with an activating agent present at a mitochondrion to form an activated product that inhibits or enhances the opening of a mitochondrial permeable transition pore (MPTP).

Most activated compounds that inhibit or enhance the opening of the MPTP, including α-oxoaldehydes and α-oxoketones, as well as aldehydes and ketones in general, cannot be made by existing prodrug technologies, which are more limited to targeting enzymes and undergoing traditional enzymatic hydrolysis reactions, such as the cleavage of peptides, esters, and others, to create alcohols, carboxylic acids, amines, amides, thiols, among others. Using prodrug technology, these activated molecules can be created directly inside the mitochondrial matrix, e.g., inside the matrix on the surface of the inner mitochondrial membrane—the same location as the MPTP—nontarget effects, e.g., toxicity or side effects, would be reduced considerably, and the health status of mitochondria can be altered very quickly.

The MPTP is an extremely important target for the treatment of diseases because upon opening it allows the rapid fluctuation changes of Ca⁺² in and out of the mitochondrial matrix. This causes changes in concentration gradients, depolarization of the mitochondrial membrane potential, decreases in energy production, mitochondrial swelling, and the release of apoptotic proteins, such as cytochrome c, into the rest of the cell initiating necrosis or apoptosis. Also, MPTP is not present in all cells, but is usually formed in the inner mitochondrial membrane upon traumatic injury or as a result of disease. Increased ROS levels lead to an influx of Ca⁺² into the mitochondrial matrix, which leads to MPTP formation from its components voltage-dependent anion channel (VDAC), adenine nucleotide translocator (ANT), cyclophilin D (CypD, and other molecules (Rao et al. 2014). Upon binding of methylglyoxal to MPTP, not only was it shown to close the MPTP channel, but no other negative side effects were shown in the cell (Speer et al. 2003), which shows that compounds provided herein provides patients with an effective treatment mechanism for various acute and chronic pathological diseases and conditions, without the deleterious side effects caused by nonspecific and reactive medications.

In some embodiments, the inactive compound comprises a dienol ether, a divinyl thioether, a dienamine, an enol ether, a vinyl thioether, an enamine, or a combination thereof. As described in WO 2016/023015, WO 2017/049305 and WO 2018/102463, these moieties are reactive with various activating agents, e.g., free radicals, ROS, and other reactive species, to form activated compounds.

The effectiveness of the prodrug can be increased by having more than one reactive moiety, e.g., dienol ether, divinyl thioether, or dienamine, on the compound. Additionally, more than one therapeutic active product, either the same product, or different products, can be released by reaction with the activating agent. In some embodiments, this is achieved with a polymeric prodrug comprising multiple units that are activated by cleavage of each unit from the polymer. See, e.g., the polymers and multifunctional compounds in WO 2016/023015, WO 2017/049305 and WO 2018/102463.

A benefit of using enol ethers as the reactive moiety is that enol ethers are already common functional groups that are present in numerous mammalian (including human) tissues. These groups are located on plasmalogens, which are phospholipids found in high concentrations in mammalian nervous, immune, and cardiovascular systems (Braverman and Moser, 2012). The prodrug compounds provided herein are only reactive to high-energy reactive chemical species, such as hydroxyl radicals, hydrogen peroxide, and superoxide.

The reactivity of the prodrug compounds provided herewith can be modified by adding another electron-donating group to the other side of the enol ether, or derivatives of it, increasing the electron-density donation to the reactive alkene group, making it more sensitive to the reactive species. Other ways to modify the reactivity of the compounds are provided in WO 2017/049305 and WO 2018/102463, incorporated by reference.

As an example, by having the dienol ether, and its derivatives, form a cyclic structure, such as a heterocyclic 5-membered ring where one carbon is attached to two oxygen atoms on the other side, the reactivity to the ROS can be increased considerably after the initial ROS attack. This is because the electron transfers from each bond will form a cyclic shape, resulting in the formation of another byproduct. Formaldehyde can be formed as one of the byproducts, which adds significant value with this technology because glutathione, an intramitochondrial matrix antioxidant, attacks aldehydes such as formaldehyde as well as the dicarbonyl groups. The formation of another aldehyde byproduct competitively competes with the prodrug dicarbonyl to bind to the glutathione. By also creating a competitive inhibitor, the half-life of our activated product can be increased, giving it more time to bind to the MPTP. The formation of extra aldehydes and ketones can allow a decrease of the therapeutic dosage of the prodrugs by potentially one-half, increasing the safety and non-toxicity properties considerably.

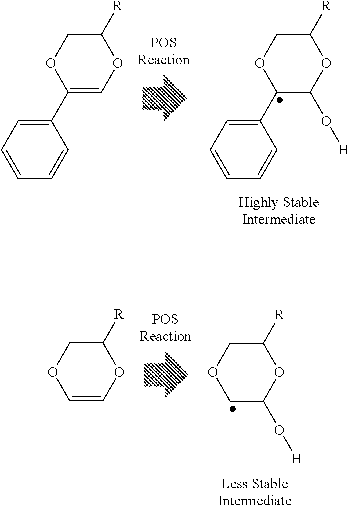

The reactivities of the compounds provided herein with reactive species can be altered by adding conjugation or eliminating conjugation. For example, the structure above and to the left has a conjugated aromatic to it. This is extremely stable for a free radical, and the more stable a radical will be on a structure, after the initial free radical reaction, the faster it will happen. Therefore, adding an aromatic conjugated to the divinyl ether increases the sensitivity of the prodrug to the reactive species-sensitive alkene. Although there are several alkenes on the benzene ring, it is an aromatic, so the free radical will not be able to bind to an oxygen atom. The only carbons that are oxidized are the carbons that are part of the reactive species-sensitive alkene attached to the oxygen atoms.

On the right side, glyoxal can be produced where there is no other functional groups attached. This might decrease the sensitivity slightly, but that is desired if a specific reactive species is targeted.

It is noted that the above two compounds have no α-hydrogen atoms on adjacent carbons. Free radicals attack α-hydrogens next to alkenes since the bond dissociation energy is low on those carbons as a result of resonance. By removing them, pure compounds are created upon reactive species attack where the only reactions that happen are epoxidation and free radical addition reactions to alkenes, and not hydrogen abstraction reactions which potentially can cause other byproducts.

In various embodiments, the activating agent is a free radical, a reactive oxygen species, a reactive lipid species, another reactive species, or an enzyme present in the mitochondria.

In many of these embodiments, and as further elaborated in WO 2017/049305 and WO 2018/102463, the activating agent is a free radical, a reactive oxygen species, a reactive lipid species, or another reactive species. These reactive species will be reduced in the reaction.

In other embodiments, the activating agent is electromagnetic radiation, where the prodrug has a light absorbing group (e.g., a dye) that, upon stimulation with a particular wavelength of light modifies the prodrug to form the MPTP modifying active agent, or to create a modified prodrug that reacts with an enzyme, a free radical, an ROS, a reactive lipid species, or another reactive species to form the MPTP modifying active agent. See WO 2018/102463 for a further elaboration of these embodiments.

In additional embodiments, the activating agent is an enzyme, as are utilized with other prodrugs. Nonlimiting examples of enzymes that can be utilized are carboxylesterase, acetylcholinesterase, esterase, butyrylcholinesterase, paraoxonase, matrix metalloproteinase, alkaline phosphatase, β-glucuronidase, human valacyclovirase, hydrolase, plasmin, protease, prostate-specific antigen, purine-nucleoside phosphorylase, carboxypeptidase G2, penicillin amidase, β-lactamase, β-galactosidase, cytosine deaminase, serine hydrolase, cholinesterase, phosphorylase, acetaldehyde dehydrogenase, dehydrogenase, aldehyde oxidase, amino acid oxidase, oxidase, P450 Reductase, DT-diaphorase, thymidine phosphorylase, glutathione S-transferase, deoxycytidine kinase, lyase, thymidylate synthase, tyrosinase, methionine δ-lyase, cytosine deaminase, β-lactamase, penicillin amidase, carboxypeptidase, β-glucuronidase, thymidine kinase, and nitroreductase. In some embodiments, the enzyme is an oxidoreductase such as aldehyde oxidase, amino acid oxidase, cytochrome P450 reductase, DT-diaphorase, cytochrome P450, or tyrosinase; a class 2 transferase such as thymidylate synthase, thymidine phosphorylase, glutathione S-transferase, or deoxycytidine kinase; or a class 3 hydrolase such as carboxylesterase, alkaline phosphatase, or β-glucuronidase. An ADEPT, VDEPT or GDEPT approach can also be utilized (Rooseboom et al., 2004).

A potential benefit to this approach is that some enzymes, proteins, biological activating agents might be heightened or decreased in diseased versus healthy cells, or between different types of cells in general that can give us greater specificity towards our target location. For example, in Alzheimer's patients, there is a substantial decrease in acetylcholinesterase activity in the brain (Garcia-Ayllón 2011). As such, providing a prodrug that is activated by acetylcholinesterase would be unlikely to target cells in the brain. On the same note, the same can be said about different areas in the body and cells that might experience higher or lower reactive species, and we can use the same type of approach to target different locations as well.

By using an enzymatic or hydrolytic approach, prodrugs can be activated through other means. These cleavable groups can include, but are not limited to, esters, amides, thioesters, formyls, thioformyls, and formamides. The cleavable bonds that can be broken can include, but are not limited to C—N, O—C, S—C, and P—C.

The following diagrams show the reaction mechanism of two compounds that are activated by enzymes.

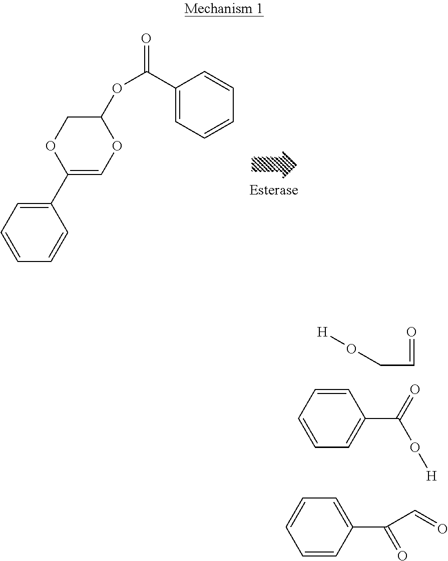

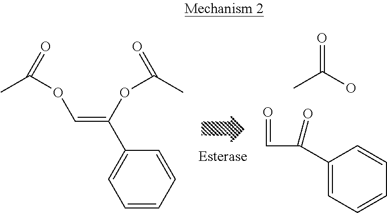

Mechanism 1 has an enzymatic esterase cleavage that will quickly form an aldehyde group, which will cause the resonance shifting of electrons to form the phenylglyoxal compound. Mechanism 2 has two ester groups that can be cleaved by esterases to form the MPTP targeting group, in this case phenylglyoxal.

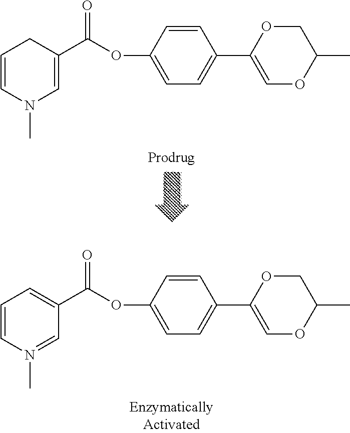

The activated product above, after esterase cleavage, forms p-hydroxyphenylglyoxal, which irreversibly activates the MPTP. This prodrug is designed to cross the blood-brain barrier to treat tumors.

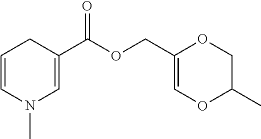 [SMILES: CC1COC(COC(=O)C2=CN(C)C=CC2)=CO1]

Another prodrug structure is provided above that is designed to cross the blood-brain barrier, but upon activation and further esterase cleavage, forms 3-hydroxyglyoxal, an MPTP activation molecule.

In various embodiments, the prodrug, upon activation, forms, in addition to the active product that modifies the MPTP, another useful product, for example another therapeutic molecule. For example, the prodrug could take any medication, now known or later discovered, and activate both it and the MPTP-inhibiting or -enhancing compound at the same time. This would allow for one or multiple therapeutic pathways to be activated simultaneously, for better treatment and broader therapeutic coverage, and a more robust treatment route. The additional activated compound(s) could allow for, and not limited to, increased penetration, increased accumulation in desired cellular, subcellular, and human body locations, increased efficacy through one or more therapeutic pathways, and/or other means. An example of this, how it would be useful, it to create a prodrug to dopamine, which cannot cross the blood brain barrier on its own, as a result of its high polarity. The prodrug could mask the hydroxyl group hydrogens, and create them once across the blood brain barrier and inside the mitochondria of the neurons. This can allow for both better efficacy for both our compounds and other medications, where they will benefit each other from transport to therapeutic activity. Examples of medications that can be products of any of the compounds provided herewith is provided at pp. 26-42 of WO 2017/049305, incorporated by reference.

An active product of these embodiments can be any compound that inhibits or enhances the opening of the MPTP. Examples of such products include those described in Eriksson et al., 1998; Speer et al., 2003; Johans et al., 2005; Linder et al., 2001; Sileikyte et al., 2015; Bhutia et al., 2016; and Sun et al., 2014. Thus, in some embodiments, the activated product is an α-dicarbonyl, e.g., as described in Eriksson et al, 1998; Speer et al., 2003; Johans et al., 2005; and Linder et al., 2001. These particular aldehyde and ketone α-dicarbonyl products specifically target the mitochondrial pore on the matrix side, and bind to its specific arginine residue, by covalent or non-covalent interactions, depending on the activated compound. Nonlimiting examples of these α-dicarbonyls are glyoxal, methylglyoxal, diacetal, acetyl propionyl, acetoin, benzil, phenylglyoxal, an α-hydroxy ketone, an α-hydroxy aldehyde, a dione, an α-ketone aldehyde, and α-chloro ketone, an α-chloro aldehyde, p-hydroxyphenyl glyoxal, or a substituted phenyl glyoxal, depicted below, along with similar compounds, any of which could be utilized as a product of the invention compositions.

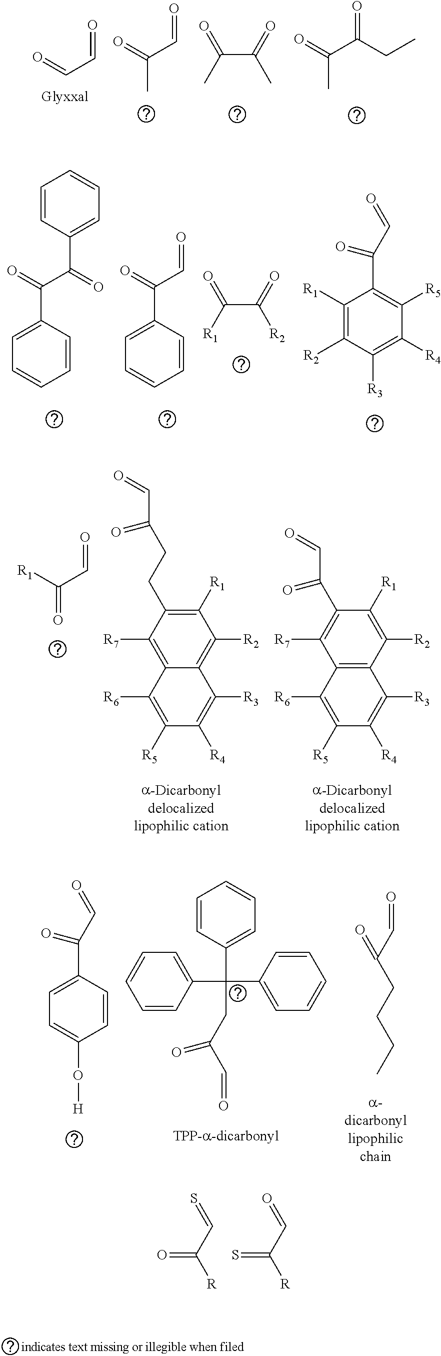

An example of a particularly useful α-dicarbonyl in these embodiments is methylglyoxal. Methylglyoxal incubation with isolated mitochondria show complete suppression of permeability transition by covalently binding to the MPTP (Speer et al., 2003). Glyoxal also exhibits covalent binding. The methylglyoxal reaction is rapid suppression of the permeability transition occurred within 5 min. The binding and suppressing of MPTP opening is very selective and specific since no other mitochondrial function is disturbed by this process. The reaction is reversible, returning the transitional pore to its native state over time (Speer et al. 2003).

The compounds of these embodiments can be designed to target the MPTP without targeting lipid peroxidation. This is achieved by designing compounds water soluble, or have bulky groups, that won't allow it to effectively insert into the lipid bilayer. Therefore, the only reactions that will take place will be in the polar media of the mitochondria or elsewhere in the cell. Below are two examples of these types of molecules.

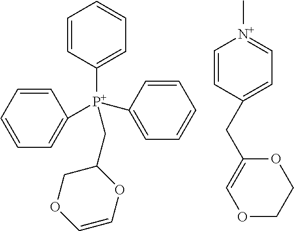 [SMILES: C1=COC(C[P+](c2ccccc2)(c2ccccc2)c2ccccc2)CO1 ; C[n+]1ccc(CC2=COCCO2)cc1]

The molecule on the left has a bulky triphenylphosphonium group, and a small chain, preventing it from inserting into the lipid bilayer. The product of the reaction of that compound with a reactive species is glyoxal, inhibits opening of the MPTP. The compound on the right is very water soluble, and, upon reaction with a reactive species, produces a highly polar—dicarbonyl that activates the MPTP, causing mitochondrial dysfunction, a potential cancer treatment.

The molecule below already has a permanent cationic charge, so it will not cross the skin or BBB. As a result of its polar hydroxyl group, it will mainly associate in the mitochondrial matrix, and upon reactive species reaction, form a MPTP opening 4-hydroxylphenylglyoxal group.

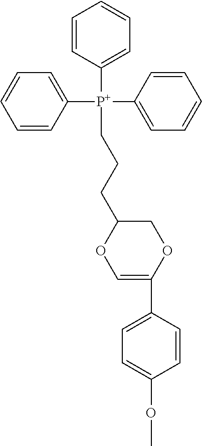 [SMILES: COc1ccc(C2=COC(CCC[P+](c3ccccc3)(c3ccccc3)c3ccccc3)CO2)cc1]

### Mitochondrial Delivery Vectors

Delivery of the active products can result from a prodrug compound where the active product is attached onto naturally-occurring molecules, and their analogues, that are safe, naturally metabolized and/or delivered to specific parts of the body. An example of this is riboflavin, which travels across the blood brain barrier through pores and reaches into the mitochondrial matrix. With a prodrug comprising riboflavin or other molecules associated with energy production that is released when the inactive compound reacts with the activating agent, those areas in the body and cells can be targeted, providing additional specificity to our targeted approach.

A mitochondria-specific targeting approach can also be utilized (Frantz and Wipf, 2010; Eirin et al., 2017), including with particles (Wongrakpanich et al., 2014).

In some embodiments, a delocalized lipophilic cation, such as a triphenylphosphonium moiety, is utilized on the compounds to aid in the delivery of compounds to the mitochondrial matrix (Murphy 2008). These cations are attracted to the negative charge found within the matrix as a result of the hyperpolarized mitochondrial membrane potential, while its lipophilic properties allow it to pass through the membranes. Using this approach, large doses of these cations can accumulate in targeted location inside the mitochondrial matrix. Further, many diseases have increased hyperpolarized mitochondrial membrane potentials, such as in cancer cells and cancer stem cells. Using this approach, the mitochondrial matrix is selectively targeted in diseased and cancerous cells over normal cells, for more specific and effective treatments with less side effects (Wong et al., 1995; Ye et al., 2011).

Furthermore, these delocalized lipophilic cations localize on the lipophilic inner mitochondrial membrane, on the matrix-side, which is the same location as the target arginine residue-binding site on the MPTP. This is shown with antioxidant molecules such as mitoquinone and its derivatives, that not only accumulate significantly in the mitochondria, but adsorb onto the surface of the inner mitochondrial membrane—matrix side—with their lipophilic tails becoming embedded in the bilayer (Sheu et al. 2006). By using delocalized lipophilic cations diseased cells can be targeted, and the prodrugs, and their corresponding activated products are delivered directly to the sites of the MPTP (Ross et al. 2006; Severina et al. 2007).

Additionally, delocalized lipophilic cations, are secreted over time from the cell and excreted from the body, so permanent depolarization of the cell after treatment with a delocalized lipophilic cation, or the accumulation of these therapeutic agents inside the body over time, is of minimal concern. In fact, when a test group was administered a blood-brain barrier permeable delocalized lipophilic cation daily for a year, no signs of toxicity was exhibited throughout the tests, or afterwards (Ross et al. 2006; Severina et al. 2007; Snow 2010).

Specific examples of mitochondrial targeting compounds include the following compounds that are delocalized lipophilic cations. The two molecules on the right upon an ROS attack can form two activated therapeutic molecules at once.

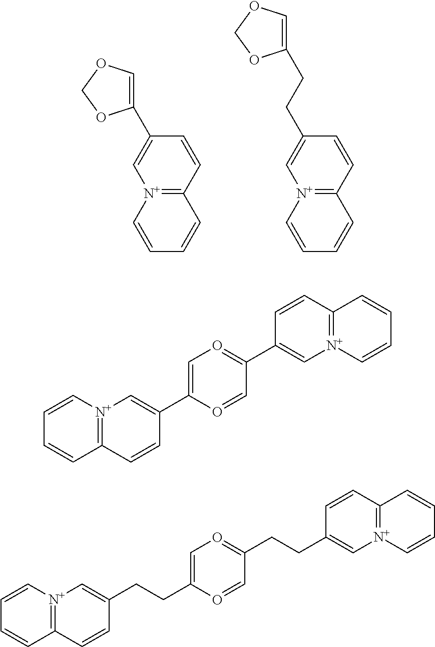

Specific examples of mitochondrial targeting compounds include the following molecules that go from a positive charge to a negative or neutral charge upon ROS reaction in the mitochondria matrix :

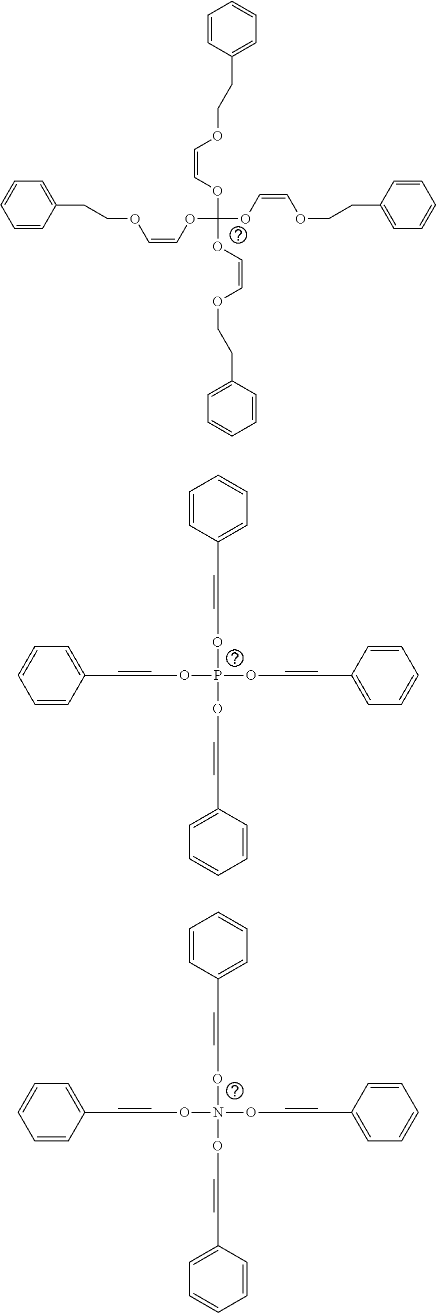

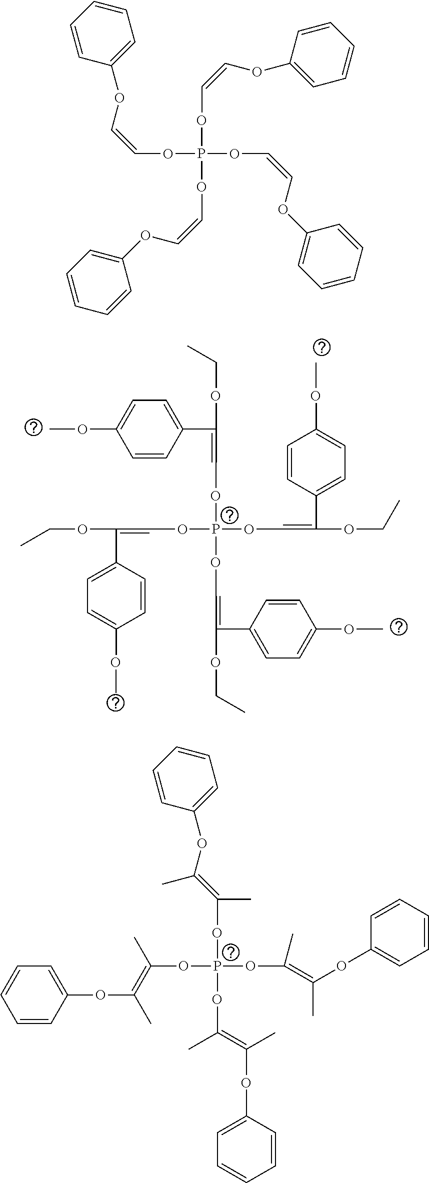

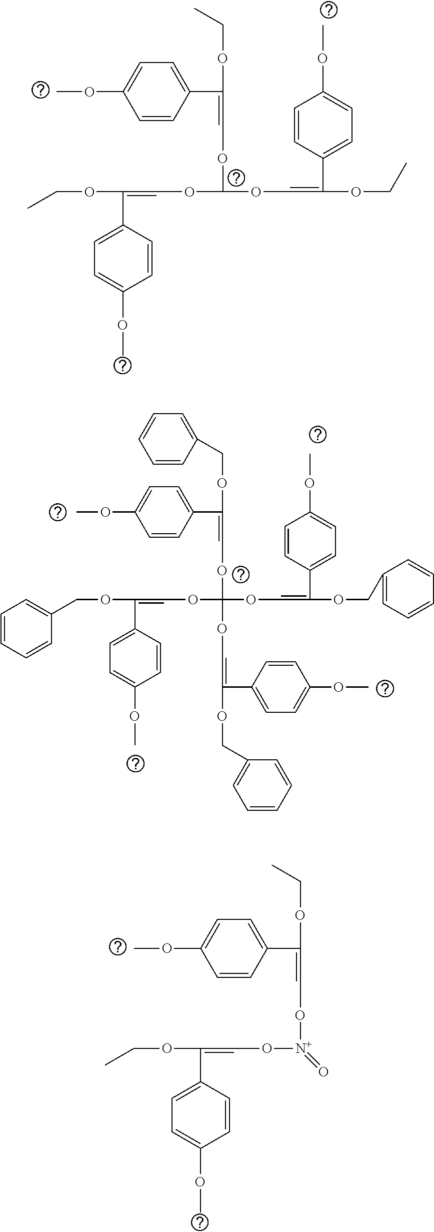

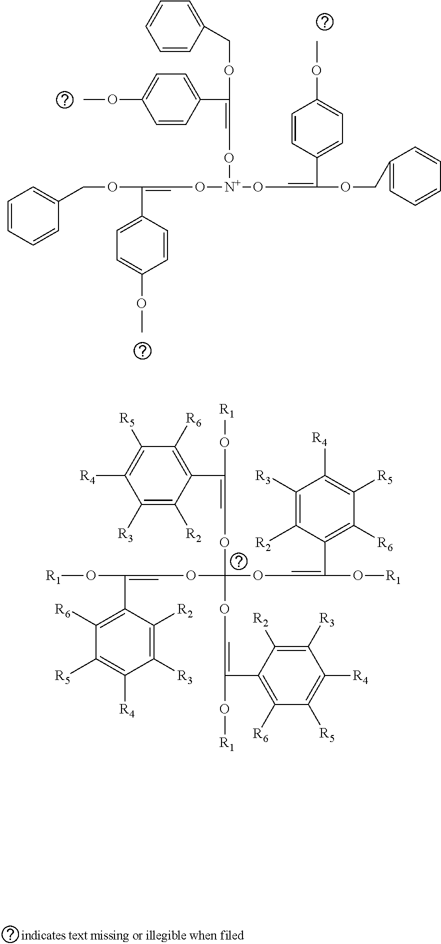

The following molecules go from a neutral charge to a negative charge upon ROS reaction. Since they are neutral, they will not specifically target the mitochondrial matrix, but can pass through membranes and can go to many different locations in the cell and bloodstream.

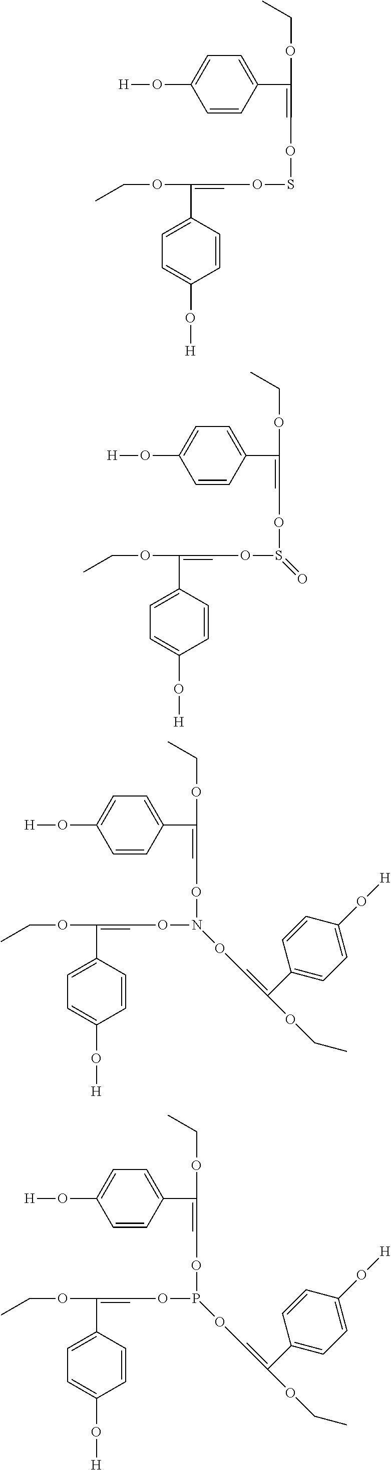

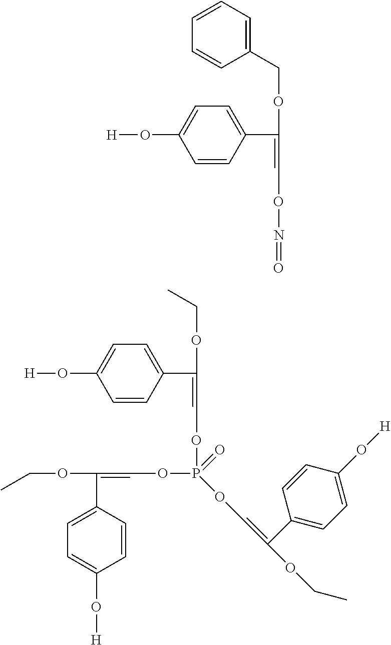 [SMILES: O=NOC=C(OCc1ccccc1)c1ccc(O)cc1 ; CCOC(=COP(=O)(OC=C(OCC)c1ccc(O)cc1)OC=C(OCC)c1ccc(O)cc1)c1ccc(O)cc1]

The following molecules are attached to drug delivery vehicles, including riboflavin, FMN, and FAD analogs that have reactive species-sensitive groups attached and will be brought through membranes and the blood-brain barrier. Riboflavin, FMN, and FAD are used in oxidative phosphorylation found in the mitochondrial matrix, which allows these molecules to target energy production.

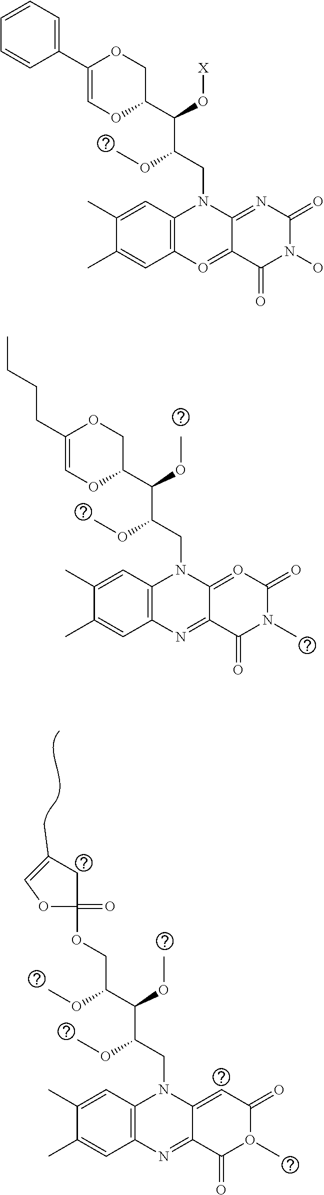

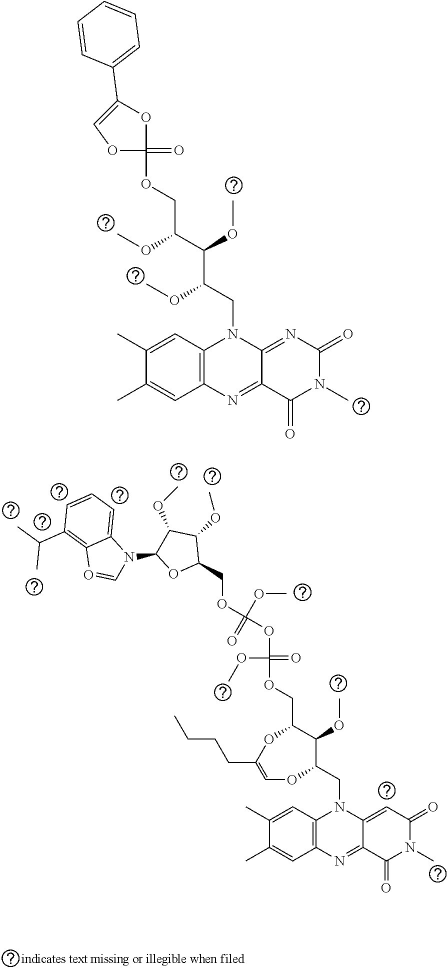

Specific examples of mitochondrial targeting compounds include the following molecules that are similar in structure to mitoquinone, a known inner mitochondrial membrane targeting/adsorbing molecule. Our prodrugs should exhibit similar binding to the inner mitochondrial membrane properties as mitoquinone. One structure is a pore opening prodrug since it forms a soluble alpha-dicarbonyl moiety upon reactive species reaction, whereas the other is a pore closing prodrug since it forms a lipophilic and highly non-polar activated agent upon reactive species reaction

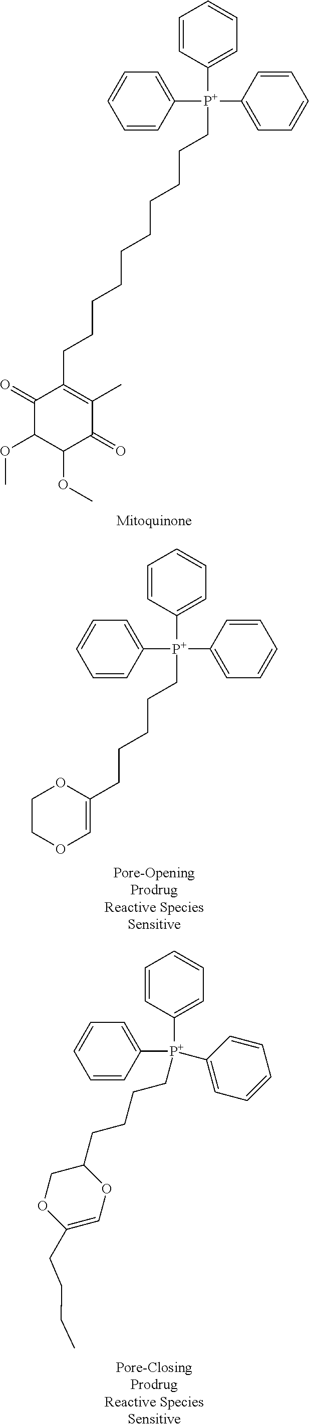 [SMILES: COC1C(=O)C(C)=C(CCCCCCCCCC[P+](c2ccccc2)(c2ccccc2)c2ccccc2)C(=O)C1OC ; C1=C(CCCCC[P+](c2ccccc2)(c2ccccc2)c2ccccc2)OCCO1 ; CCCCC1=COC(CCCC[P+](c2ccccc2)(c2ccccc2)c2ccccc2)CO1]

The following molecule is an example of creating both an α-dicarbonyl activated agent as well as another type of medication. In this case, dopamine is incorporated into the molecule and is released on reaction of the molecule with a reactive species. Dopamine cannot cross the blood brain barrier, so this is an example of our technology to help deliver other molecules across the blood brain barrier that would not do so otherwise. After it crosses the blood brain barrier, the

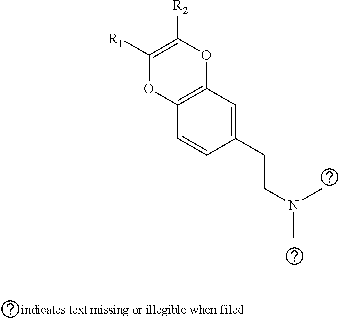

prodrug will be activated in the mitochondria which will create a α-dicarbonyl MPTP regulating agent, as well as dopamine which is used as a neurotransmitter.

The following molecules are examples of general cyclic prodrugs, which can be utilized in compounds of the present invention to target the MPTP and/or reactive species.

### Cyclic Prodrug Examples

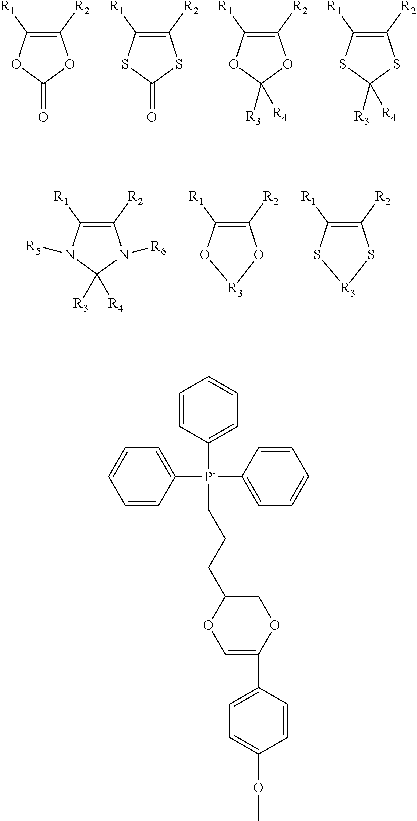

An additional compound is provided above that, upon reaction with a reactive species, creates an MPTP inhibitor.

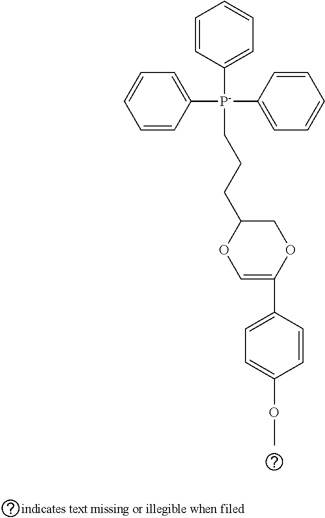 [SMILES: COc1ccc(C2=COC(CCC[P-](c3ccccc3)(c3ccccc3)c3ccccc3)CO2)cc1]

The above compound, upon reaction with a reactive species, produces a product that is an MPTP activator.

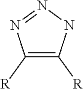

The compound above is designed to form nitrogen gas after reacting with a reactive species, while the nitrogen atoms in the unreacted compounds donate electron density to the alkene, making the compound more reactive.

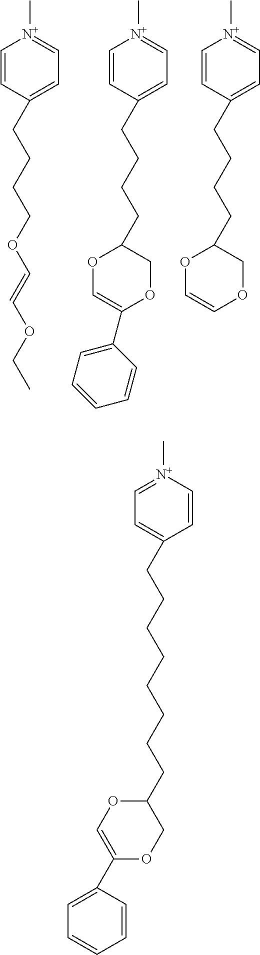 [SMILES: CCO/C=C/OCCCCc1cc[n+](C)cc1 ; C[n+]1ccc(CCCCC2COC(c3ccccc3)=CO2)cc1 ; C[n+]1ccc(CCCCC2COC=CO2)cc1 ; C[n+]1ccc(CCCCCCCCC2COC(c3ccccc3)=CO2)cc1]

The above compounds enter the mitochondrial inner membrane and react with reactive species (e.g., a reactive lipid species such as cardiolipin hydroperoxide or cardiolipin peroxyl radical) to create an MPTP binding molecule while reducing the lipid peroxyl group. The compound on the far left produces glyoxal and ethanol; the second compound produces phenylglyoxal; the third glyoxal. Notice the comparison in structures from the first and third, where they both produce the same MPTP group, but the third compound cyclic structure, and therefore does not produce an additional molecule in the reaction, while the first produces ethanol.

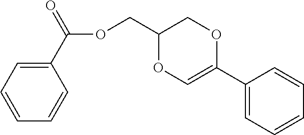 [SMILES: O=C(OCC1COC(c2ccccc2)=CO1)c1ccccc1]

The structure above produces phenylglyoxal, benzoic acid, and glycerol after reacting with a reactive species. This compound has a longer chain than the others which allows it to reach the further down hydroperoxides and peroxyl lipid radicals.

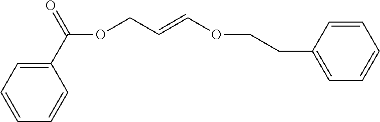 [SMILES: O=C(OC/C=C/OCCc1ccccc1)c1ccccc1]

The above compound produces phenylethyl alcohol, benzoic acid, and glyceraldehyde after reaction with a reactive species.

On all of these, while they are designed to remove the lipid peroxyl groups, they can also react with free-flowing reactive species such as perhydroxyl radicals, which are just as important since those initialize lipid peroxidation free radical reactions. Therefore these compounds are designed to target and terminate the initiation and propagation steps in the free radical chain reaction process, as does the plasmalogen vinyl ether group.

### Mechanisms

Without being bound to any particular mechanism, it is believed that the following describes the mechanisms of the embodiments described herein.

### Mechanism of Pore-Targeting Prodrugs

The following mechanisms are examples of biologically and/or enzymatically cleavable prodrugs that are then activated to form MPTP-targeting alpha dicarbonyl therapeutic agents. This mechanism has resonance causing the displacement of the other acetyl group that incorporates a hydrolysis reaction to produce the desired alpha-dicarbonyl activated therapeutic agent.

The above mechanisms for compounds of the present invention show mitochondria targeting, particularly its specific hydroxyl radicals, superoxide, hydrogen peroxide, and other reactive species in the inner mitochondrial membrane or elsewhere. These illustrations provide a dienol ether on the top, and a cyclic dienol ether on the bottom, and another cyclic structure with two sets of dienol ethers. The oxygen atom on the dienol ethers can be substituted with N, S, or P, or combinations thereof. The dienol ethers are useful for forming α-dicarbonyls.

The non-cyclic Mechanism 1 forms alcohols. In the cyclic mechanism in Mechanism 2, the hydrolysis reaction will cause a quick shift in electron density in a cyclic pattern that forms formaldehyde as the other byproduct. Mechanism 3 shows the creation of two activated dicarbonyl groups with one reactive species reaction, followed by hydrolysis.

The above illustration shows epoxide formation with cyclic structures, creating carbon dioxide, or molecules such as carbonyl sulfides, as well as α-dicarbonyls and carbonyl sulfide derivatives, which us used to alter the reactivity, stability, and efficacy of specific amino acid targeting compounds, e.g., lysine residues. This compound can be designed to use a hydrogen abstraction mechanistic route, where a reactive species causes a resonance reaction that produces α-dicarbonyl formation. One example of this is shown above. This hydrogen is placed directly on the enol ether group, or be part of an aromatic group, where resonance cascades to the enol ether, or derivatives, to form the corresponding glyoxal derivatives.

### Mechanism for Mitochondrial Charge Targeting Embodiments

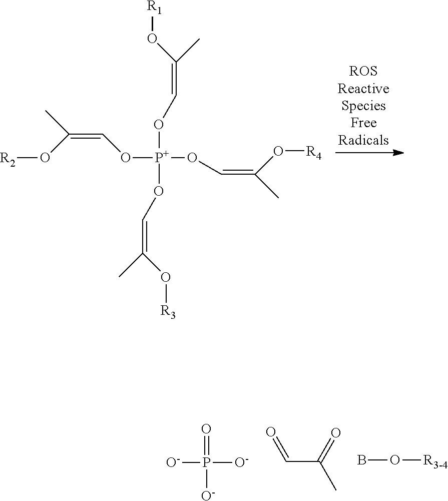

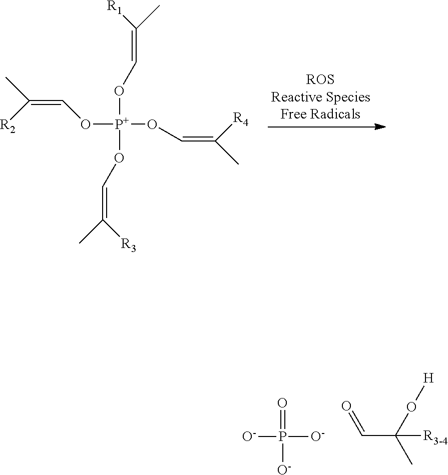

The top mechanism above shows a dienol ether, for example a dienamine or divinyl thioether, or a combination thereof, that reacts with reactive species to produce phosphate, methylglyoxal, and an alcohol. The reactive species most abundant in the mitochondrial matrix are superoxide, hydrogen peroxide, and hydroxyl radicals.

The bottom mechanism shows an enol ether, which could be substituted with an enamine or a vinyl thioether. Upon reactive species or free radical reaction, it forms a phosphate, and an α-hydroxyl aldehyde.

In these examples, a phosphonium+1 cationic charge went to a (−3) anionic phosphate charge upon reactive species or free radical reaction. This prodrug technology can thus break positive charges down after the prodrug is delivered inside the mitochondrial matrix. After reaction, they can form safe, and non-toxic molecules, such as phosphate, or therapeutic compounds, such as thiophiosphate for cancer treatment. Breaking down the cationic charge into negative charges prevents a buildup of cations in the matrix which could affect membrane polarization. Increase in the buildup of anionic charge can also be achieved, restoring polarization. Beneficial phosphates, sulfates, and nitrates can be produced can help benefit the cell as well. Nitrites become oxidized to nitrates, which undergo further chemical reactions to produce nitric oxide, an important molecule for the functioning of the body. Neutral charges can also be converted into negative charges.

### Pharmaceutical Applications

A myriad of diseases involve mitochondrial dysfunction, characterized by high levels of ROS production, low levels of antioxidants, increased Ca⁺² influx, and decreased levels of ATP production (Görlach and Bertram, 2015). Examples of diseases where mitochondrial dysfunction include neurodegenerative disease and stroke (Prentice et al., 2015), pancreatitis (Maleth and Higyi 2016), ischemia (Motloch et al., 2015), ischemia-reperfusion injury (Javadov et al., 2017) and pancreatic cancer (Sun et al., 2014) and many others (Ponnalagu and Singh 2017). Indeed, mitochondrial dysfunction is implicated in aging (Wagner and Payne 2011; Rottenberg and Hoek 2017). Treating these diseases with any of the above compounds reduces mitochondrial dysfunction advantageously.

Thus, also provided herein is a method of treating a mitochondrion in a patient comprising a mitochondrial dysfunction. The method comprises contacting the mitochondrion with any of the above-identified compounds. In some embodiments, the patient has diabetes, excessive aging, pancreatitis, cardiac disease, neurodegenerative disease, Alzheimer's disease, Parkinson's disease, Huntington disease, amyotrophic lateral sclerosis, multiple sclerosis, a muscular dystrophy, or has had a stroke. In some of these embodiments, the patient has heart failure, or ischemic disease, has had a myocardial infarction, or is at risk for ischemia-reperfusion injury.

More generally, since optimizing mitochondrial health is advantageous for treating any disease, the patient in these embodiments has any of the following diseases: Hemophilia, Hepatitis A, AAA (Abdominal Aortic Aneurysm), AAT (Alpha 1 Antitrypsin Deficiency), AATD (Alpha 1 Antitrypsin Deficiency), Abdominal Adhesions (Scar Tissue), Abdominal Aortic Aneurysm, Abdominal Cramps (Heat Cramps), Abdominal Hernia (Hernia), Abdominal Migraines, Abdominal Pain, Abnormal Biorhythms, Abnormal Heart Rhythms (Heart Rhythm Disorders), Abnormal Liver Enzymes, Abnormal Vagnial Bleeding (Vaginal Bleeding), Abscesses, Skin (Boils), Absence of Menstrual Periods (Amenorrhea), Abulia, Accumulation of Fluid (Ascites), Acetaminophen Liver Damage (Tylenol Liver Damage), Achalasia, Aches, Achondroplasia, Achondroplastic Dwarfism (Achondroplasia), Acid Reflux (Gastroesophageal Reflux Disease (GERD)), Acid Reflux, Acne Cystic (Boils), Acne Rosacea (Rosacea), Acne Scars (Scars), Acne, Acquired Brain Injury and Damage, Acquired Bronchiectasis (Bronchiectasis (Acquired, Congenital)), Acquired Epileptic Aphasia (Landau-Kleffner Syndrome), Acquired Hydrocephalus (Hydrocephalus), Acquired Immunodeficiency Syndrome (AIDS), Acrochordon (Skin Tag), ACTH-dependent Hypercortisolism (Cushing's Syndrome), ACTH-independent Hypercortisolism (Cushing's Syndrome), Actinic Keratosis, Acustic Neuroma (Vertigo), Acute and Chronic Bursitis, Acute Bacterial Prostatitis (Prostatitis (Inflammation of the Prostate Gland)), Acute Compartment Syndrome (Compartment Syndrome), Acute Diseases and Conditions, Acute granulocytic leukemia, Acute Hepatitis B (Hepatitis B), Acute Intermittent Porphyria (Porphyria), Acute Kidney Failure (Kidney Failure), Acute Lung Injury (ARDS), Acute Lymphocytic Leukemia (Leukemia), Acute myelogenous leukemia, Acute Myeloid Leukemia, Acute Pancreatitis (Pancreatitis), Acute Porphyria (Porphyria), Acute Respiratory Distress Syndrome (ARDS), Acute Valley Fever (Valley Fever), Ad14 (Killer Cold Virus (Adenovirus Infection, Ad14)), ADD (ADHD in Children), Addiction, Addison Anemia (Pernicious Anemia), Addison Disease, Adenocarcinoma, Adenomatous Polyposis Coli (Gardner Syndrome), Adenomatous Polyposis of the Colon (Gardner Syndrome), Adenomyosis (Uterine Fibroids), Adenosarcoma, Adenovirus Infection, ADHD, Adrenal cancer, Adrenal Gland Tumor (Pheochromocytoma), Adrenal Insufficiency (Addison Disease), Adrenal Pheochromocytoma (Pheochromocytoma), Adrenocortical carcinoma, Adult Acne (Rosacea), Adult Onset Diabetes (Diabetes Mellitus), Adult Onset Still (Still's Disease), Aganglionosis (Hirschsprung Disease), Age Spots (Freckles), Age-Related Macular Degeneration (Macular Degeneration), Ageusia (Taste Disorders), Agnosia, Facial (Face Blindness (Prosopagnosia)), Agoraphobia, Agranulocytosis (Neutropenia), Agraphia, AIDS, Air Sick (Motion Sickness (Sea Sickness, Car Sickness)), AKU (Alkaptonuria), ALAD Porphyria (Porphyria), Albinism (Birthmarks and Other Skin Pigmentation Problems), Alcaptonuria (Alkaptonuria), Alcohol Abuse and Alcoholism, Alien hand syndrome, ALK (Keratoplasty Eye Surgery (ALK)), Alkaptonuria, Allan-Herndon-Dudley syndrome, Allergic Conjunctivitis (Pink Eye), Allergic Granulomatosis And Angiitis (Churg-Strauss Syndrome), Allergic Purpura (Henoch-Schonlein Purpura), Allergic Reaction (Anaphylaxis), Allergic Rhinitis (Allergy), Allergic Rhinitis (Hay Fever), Allergies, Allergy, Eczema (Atopic Dermatitis), Allergy, Eye (Eye Allergy), Allergy, Food (Food Allergy), Allergy, Insect (Insect Sting Allergies), Allergy, Latex (Latex Allergy), Allergy, Plant Contact (Poison Ivy, Oak, and Sumac), Alopecia Areata, Alpha 1 Antitrypsin Deficiency, Alpha Thalassemia, Alpha-1 Proteinase Inhibitor (Alpha 1 Antitrypsin Deficiency), Alpha-1 Related Emphysema (Alpha 1 Antitrypsin Deficiency), Alpha-galactosidase Deficiency (Fabry's Disease), Alport Syndrome, ALS (Amyotrophic Lateral Sclerosis), Alveolar Osteitis (Dry Socket), Alveolus Cancer (Oral Cancer), Alzheimer's disease, AMA (Antimitochondrial Antibodies), Amaurosis fugax, Amblyopia, Amenorrhea, American Trypanosomiasis (Chagas Disease), AML (Leukemia), Ammonia Dermatitis (Diaper Rash), Ammonia Rash (Diaper Rash), Amnesia, Amniocentesis, Amyloidosis, Amyotrophic Lateral Sclerosis, ANA (Antinuclear Antibody), Anal Cancer, Anal Fissure, Anaphylactoid Purpura (Henoch-Schonlein Purpura), Anaphylaxis, Anaplastic astrocytoma (Brain cancer), Anaplastic Carcinoma (Thyroid Cancer), Anemia, Anencephaly, Anesthesia Related Hyperthermia (Malignant Hyperthermia), Aneurysm, Angelman syndrome, Angiitis (Vasculitis), Angina Symptoms, Angioedema (Hives), Angiogram Of Heart (Coronary Angiogram), Angio-Osteohypertrophy Syndrome (Klippel-Trenaunay-Weber Syndrome), Angioplasty (Coronary Angioplasty), Angiosarcoma, Ankylosing Spondylitis, Anorexia Nervosa, Anosognosia, Anovulation (Vaginal Bleeding), Anoxia Encephalopathy (Encephalopathy), Anserine Bursitis (Knee Bursitis), Anterolisthesis (Spondylolisthesis), Anthrax, Antibiotic Resistance, Antibiotic-Caused Colitis (Clostridium Difficile Colitis), Antibiotic-Resistant Tuberculosis (Extensively Drug-Resistant Tuberculosis (XDR TB)), Antibiotic-Resistant Tuberculosis XDR-TB (Extensively Drug-Resistant Tuberculosis (XDR TB)), Anticardiolipin Antibody (Antiphospholipid Syndrome), Anti-CCP (Citrulline Antibody), Antiemetics, Anti-nausea (Antiemetics), Antinuclear Antibody, Antiphospholipid Syndrome, Antisocial Personality Disorder, Antitrypsin (Alpha 1 Antitrypsin Deficiency), Anti-vomiting (Antiemetics), Antoni's Palsy (Facial Nerve Problems), Antro-duodenal Motility Study, Anxiety, Aortic Dissection, Aortic Heart Valve Replacement (Heart Valve Disease Treatment), Aortic Stenosis, APC (Gardner Syndrome), APD (Auditory Processing Disorder in Children), Apgar Score, Aphasia with Convulsive Disorder (Landau-Kleffner Syndrome), Aphasia, Aphthous Ulcers (Canker Sores), Apnea, Sleep (Sleep Apnea), Apophysitis Calcaneus (Sever Condition), Appendectomy, Appendicitis, Appendix cancer, Apraxia, Aprosencephaly (Anencephaly), Arachnoiditis, ARDS, Areola (Breast Anatomy), Arm Cramp (Muscle Cramps), Arnold-Chiari malformation, Arrest, Cardiac (Sudden Cardiac Death), Arrhythmia (Irregular Heartbeat), ART (Infertility), Arteriosclerosis (Heart Disease (Coronary Artery Disease)), Arteriovenous Malformation, Arteritis (Vasculitis), Artery, Carotid Disease (Carotid Artery Disease), Arthralgia (Arthritis), Arthritis In Children (Juvenile Arthritis), Arthritis, Arthritis, Ankylosing Spondylitis, Arthritis, Degenerative (Osteoarthritis), Arthritis, Infectious (Septic Arthritis), Arthritis, Juvenile (Juvenile Arthritis), Arthritis, Lyme (Lyme Disease), Arthritis, MCTD (Mixed Connective Tissue Disease), Arthritis, Plant Thorn (Plant Thorn Synovitis), Arthritis, Pseudogout (Pseudogout), Arthritis, Psoriatic (Psoriatic Arthritis), Arthritis, Quackery (Quackery Arthritis), Arthritis, Reactive (Reactive Arthritis), Arthritis, Reiters (Reactive Arthritis), Arthritis, Rheumatoid (Rheumatoid Arthritis), Arthritis, Sarcoid (Sarcoidosis), Arthritis, Scleroderma (Scleroderma), Arthritis, Sjogren Syndrome (Sjogren's Syndrome), Arthritis, SLE (Systemic Lupus), Arthritis, Still (Still's Disease), Arthrocentesis (Joint Aspiration), Arthroscopy, Artificial Kidney (Hemodialysis), AS (Asperger Syndrome), Asbestosis (Asbestos-Related Disorders), Asbestos-Related Disorders, Ascending Aorta Dissection (Aortic Dissection), Ascites, Aseptic Necrosis, Asomatognosia, ASPA Deficiency (Canavan Disease), ASPD (Antisocial Personality Disorder), Asperger Disorder (Asperger Syndrome), Aspiration, Joint (Joint Aspiration), Assisted Reproductive Technology (Infertility), Asthma, Astrocytoma (Brain Tumor), Asymptomatic Inflammatory Prostatitis (Prostatitis (Inflammation of the Prostate Gland)), Ataxia, Atherosclerosis (Peripheral Vascular Disease), Atherosclerosis Prevention (Heart Attack and Atherosclerosis Prevention), Atherosclerotic Renovascular Disease (Renal Artery Stenosis), Athlete Foot, Atonic Seizure (Seizure), Atopic Dermatitis, ATR-16 syndrome, Atrial Fib (Atrial Fibrillation), Atrial Fibrillation, Atrial Flutter (Heart Rhythm Disorders), Atrial Tachycardia, Paroxysmal (Paroxysmal Supraventricular Tachycardia (PSVT)), Atrioventricular Reentrant Tachycardia (Wolff-Parkinson-White Syndrome), Atrophy, Vaginal (Vaginal Dryness and Vaginal Atrophy), Attention Deficit Hyperactivity Disorder (ADHD), Auditory Processing Disorder, Aural Hematoma (Hematoma), Autism Spectrum Disorder (ASD), Autism, Autoimmune Cholangiopathy (Primary Biliary Cirrhosis (PBC)), Autoimmune Diseases, Autoimmune Thrombocytopenic Purpura (Idiopathic Thrombocytopenic Purpura (ITP)), Autoimmune Thyroid Disease (Hashimoto's Thyroiditis), Autoimmune Thyroiditis (Hashimoto's Thyroiditis), Automatic Behavior (Narcolepsy), Autonomic Neuropathy, Diabetic (Diabetic Neuropathy), Autonomous Thyroid Nodule (Thyroid Nodules), Autosomal Dominant PKD (Polycystic Kidney Disease), Autosomal Recessive PKD (Polycystic Kidney Disease), Avascular Necrosis (Aseptic Necrosis), Avian Influenza (Bird Flu), AVM (Arteriovenous Malformation), Axillary Hyperhidrosis (Hyperhidrosis), B, Hemophilia (Hemophilia), Baby Blues (Postpartum Depression), Bacterial Arthritis (Septic Arthritis), Bacterial Endocarditis (Endocarditis), Bacterial Gastroenteritis (Gastroenteritis (Stomach Flu)), Bacterial Infections, Bacterial Vaginosis, Bad Breath, Bad Cholesterol, Baker Cyst, Balance (Vestibular Balance Disorders), Baldness (Hair Loss), Balloon Angioplasty Of Heart (Coronary Angioplasty), Balloon Endoscopy, Balloon Enteroscopy (Balloon Endoscopy), Balloon Mitral Valve (Mitral Valve Prolapse), Balloon Valvuloplasty (Heart Valve Disease Treatment), Barber Itch (Ringworm), Barium Enema, Barlow's Syndrome (Mitral Valve Prolapse), Barrett Esophagus (Barrett's Esophagus), Bartonella henselae Infection (Cat Scratch Disease), Basal Cell Carcinoma (Skin Cancer), Battle's Sign (Brain Concussion), B-Cell lymphoma (Non-Hodgkin lymphoma), BDD (Body Dysmorphic Disorder), Bee and Wasp Sting, Behavioral Disorders (Mental Health (Psychology)), Behcet Syndrome (Behcet's Syndrome), Behcet's Syndrome, Bell's Palsy (Facial Nerve Problems), Bell's palsy, Benign Prostatic Hyperplasia, Bernard-Soulier Disease, Berry Aneurysm, Beta Thalassemia, Beta-Globin Type Methemoglobinemia (Methemoglobinemia), Beuren Syndrome (Williams Syndrome), BH4 Deficiency (Tetrahydrobiopterin Deficiency), Bile Duct Cancer (Cholangiocarcinoma), Biliary Cirrhosis, Primary (Primary Biliary Cirrhosis (PBC)), Biliary Colic (Gallbladder Pain (Gall Bladder Pain)), Biliary Drainage (Duodenal Biliary Drainage), Billowing Mitral Valve (Mitral Valve Prolapse), Binswanger's Disease, Biological Therapy, Biological Valve (Heart Valve Disease Treatment), Bi-PAP (Sleep Apnea), Bipolar Disorder, Birth Defects, Birthmarks and Other Skin Pigmentation Problems, Bite, Snake (Snake Bite), Black Death (Plague), Black Eye, Black Hairy Tongue (Tongue Problems), Blackheads (Acne), Blackout (Fainting), Bladder Cancer, Bladder Incontinence (Urinary Incontinence), Bladder Infection, Bleeding Disorder (Hemophilia), Bleeding in the Eye (Subconjunctival Hemorrhage), Bleeding Nose (Nosebleed), Bleeding Varices, Bleeding, Blepharitis, Blepharospasm (Dystonia), Blindness, Blocked Lymph Vessels (Lymphedema), Blood Cancers, Blood Cell Cancer (Leukemia), Blood Clot in the Leg (Deep Vein Thrombosis), Blood Clot in the Lung (Pulmonary Embolism), Blood Clots, Blood Poisoning (Sepsis), Blood Pressure (High Blood Pressure Hypertension), Blood Pressure Of Pregnancy (Pregnancy Induced Hypertension), Blood Pressure, Low (Low Blood Pressure), Blood Transfusion, Blue Baby Syndrome (Methemoglobinemia), BMS (Burning Mouth Syndrome), Bocavirus Infection, Body Dysmorphic Disorder, Boils, Bone Cancer, Bone marrow cancer, Bone Marrow Diseases, Bone Sarcoma, Bone Spurs, Borderline Personality Disorder, Botulism, Bovine Spongiform Encephalopathy (Encephalopathy), Bovine Spongiform Encephalopathy (Mad Cow Disease), Bowel cancer (Colorectal cancer), Boxer's Ear (Cauliflower Ear), BPD (Borderline Personality Disorder), BPH (Benign Prostatic Hyperplasia), Brachial plexus injury, Bradycardia (Heart Rhythm Disorders), Brain Aneurysm, Brain Cancer, Brain Concussion, Brain Damage, Brain Hemorrhage, Brain injury, Brain Lesions (Lesions on the Brain), Brain Lesions, Brain Metastasis (Brain Tumor), Brain stem glioma (Brain cancer), Brain Tumors, Brain-Eating Amoeba (Naegleria Infection), Branchial Cyst, Breakbone Fever (Dengue Fever), Breast cancer, Broken Blood Vessel in the Eye (Subconjunctival Hemorrhage), Broken Bones, Bronchiectasis (Acquired, Congenital), Bronchitis (Acute), Bronchitis, Chronic (Chronic Bronchitis), BSE (Mad Cow Disease), Bubonic Plague, Buccal Mucosa Cancer (Oral Cancer), Buerger's Disease (Vascular Disease), Bullous Pemphigoid, Burning Mouth Syndrome, Burning Tongue Syndrome (Tongue Problems), Burns, Bursitis, Buzzing in the Ear (Tinnitus (Ringing in the Ears)), BV (Bacterial Vaginosis), C Diff (Clostridium difficile Colitis), CAD (Heart Disease (Coronary Artery Disease)), Calcium Pyrophosphate Deposition Disease (Pseudogout), Calicivirus Infection (Norovirus Infection), California Valley Fever (Valley Fever), Campomelic Dysplasia, Canavan Disease, Cancer Of Lymph Glands (Non-Hodgkins Lymphomas), Cancer of the Anus (Anal Cancer), Cancer Of The Bladder (Bladder Cancer), Cancer Of The Blood (Leukemia), Cancer of the Brain (Brain Cancer), Cancer Of The Breast (Breast Cancer), Cancer of the Cervix (Cervical Cancer), Cancer of the Colon (Colon Cancer), Cancer Of The Colon And The Rectum (Colon Cancer), Cancer Of The Endometrium (Uterine Cancer), Cancer Of The Esophagus (Esophageal Cancer), Cancer of the Gallbladder (Gallbladder Cancer), Cancer of the Head and Neck (Head and Neck Cancer), Cancer Of The Kidney (Kidney Cancer), Cancer Of The Larynx (Larynx Cancer), Cancer of the Nasopharynx (Nasopharyngeal Cancer), Cancer Of The Ovary (Ovarian Cancer), Cancer Of The Pancreas (Pancreatic Cancer), Cancer of the Penis (Penis Cancer), Cancer of the Peritoneum (Mesothelioma), Cancer of the Pleura (Mesothelioma), Cancer Of The Prostate (Prostate Cancer), Cancer of the Salivary Gland (Salivary Gland Cancer), Cancer Of The Skin (Skin Cancer), Cancer Of The Stomach (Stomach Cancer), Cancer of the Sympathetic Nervous System (Neuroblastoma), Cancer Of The Testicle (Testicular Cancer), Cancer of the Testis (Testicular Cancer), Cancer of the Thyroid (Thyroid Cancer), Cancer of the Urinary Bladder (Bladder Cancer), Cancer Of The Uterus (Uterine Cancer), Cancer of the Vagina (Vaginal Cancer), Cancer Pain, Cancer Prevention, Cancer, Canker Sores, Capgras delusion, Carcinoembryonic Antigen, Carcinoid Syndrome, Carcinoid tumors, Carcinoma of the Larynx (Larynx Cancer), Carcinoma of the Ovary (Ovarian Cancer), Carcinoma of the Thyroid (Thyroid Cancer), Cardiac Arrest, Cardiogenic Pulmonary Edema (Pulmonary Edema), Cardiolipin Antibody (Antiphospholipid Syndrome), Cardiomyopathy, Cardio-Renal, Carotid Artery Disease, Carpal Tunnel Syndrome, Cataplexy (Narcolepsy), Cataracts, Cauda Equina Syndrome, Cauliflower Ear, Causalgia (Reflex Sympathetic Dystrophy Syndrome), Causalgia, Cavernous Hemangioma (Hepatic Hemangioma), CEA (Carcinoembryonic Antigen), Celiac Disease (Gluten Enteropathy), Celiac Disease, Cellulitis, Central Nervous System Diseases, Central Nervous System Disorders, Central pain syndrome, Central pontine myelinolysis, Centronuclear myopathy, Cephalic disorder, Cephalohematoma (Newborn Jaundice (Neonatal Jaundice)), Cerebral aneurysm, Cerebral arteriosclerosis, Cerebral atrophy, Cerebral gigantism, Cerebral Palsy, Cerebral vasculitis, Cerebroside Lipidosis Syndrome (Gaucher Disease), Cervical Dysplasia, Cervical spinal stenosis, CF (Cystic Fibrosis), CFIDS (Chronic Fatigue Syndrome), Chagas Disease, Chalazion, Charcot-Marie-Tooth-Disease, Charlatanry (Quackery Arthritis), Chemical Burns (Burns), Chemotherapy, Chickenpox (Varicella), Chilblains (Frostbite), Cholelithiasis (Gallstones), Cholera, Cholescintigraphy, Cholesterol Management, Chondromalacia Patella (Patellofemoral Syndrome), Chondrosarcoma, Chordoma, Chorea, Choroiditis (Uveitis), Chronic Bronchitis, Chronic Compartment Syndrome (Compartment Syndrome), Chronic Cough, Chronic Diseases and Conditions, Chronic Fatigue Syndrome, Chronic Hepatitis B (Hepatitis B), Chronic inflammatory demyelinating polyneuropathy, Chronic Insomnia (Insomnia), Chronic Interstitial Pneumonitis (Pulmonary Fibrosis), Chronic lymphocytic leukemia, Chronic Myeloid Leukemia (Leukemia), Chronic Obstructive Lung Disease (COPD (Chronic Obstructive Pulmonary Disease)), Chronic Orthostatic Intolerance (POT Syndrome), Chronic Pain, Chronic Pancreatitis (Pancreatitis), Chronic Pelvic Pain Syndrome (Prostatitis (Inflammation of the Prostate Gland)), Chronic Renal Insufficiency (Renal Artery Stenosis), Chronic Rhinitis, Chronic Ulcerative Colitis (Ulcerative Colitis), Churg-Strauss Syndrome Circulatory Conditions, Cirrhosis, Primary Biliary (Primary Biliary Cirrhosis (PBC)), CJD (Creutzfeldt-Jakob Disease), Classical Leigh's Disease (Leigh's Syndrome (Leigh's Disease)), Claudication, Cleidocranial Dysostosis (Cleidocranial Dysplasia), Cleidocranial Dysplasia, Click Murmur Syndrome (Mitral Valve Prolapse), Clicking in the Ear (Tinnitus (Ringing in the Ears)), CLL (Leukemia), Clostridium Difficile Colitis, Clot, Blood (Blood Clots), Cluster B Antisocial Personality Disorder (Antisocial Personality Disorder), Cluster Headache, CML (Leukemia), CMT (Charcot-Marie-Tooth-Disease), CMV (Cytomegalovirus (CMV)), Coats' Disease, Coccidioidomycosis (Valley Fever), Coccydynia, Cockayne syndrome, Coffin-Lowry syndrome, Cold (Common Cold), Colitis, Colitis, Collagenous (Lymphocytic Colitis), Colitis, Crohn's (Crohn's Disease), Colitis, Lymphocytic (Lymphocytic Colitis), Colitis, Microscopic (Lymphocytic Colitis), Colitis, Ulcerative (Ulcerative Colitis), Collagen Vascular Disease (Connective Tissue Disease), Collagenous Colitis (Lymphocytic Colitis), Colon Cancer, Color Blindness, Colorectal Cancer (Colon Cancer), Coma, Combat Fatigue (Posttraumatic Stress Disorder), Compartment Syndrome, Complex Regional Pain Syndrome, Complex Tics (Tourette Syndrome), Compression of Spinal Nerves (Radiculopathy), Compulsive Behaviors, Concussion of the Brain (Brain Concussion), Concussion, Congenital Absence of Brain (Anencephaly), Congenital Aganglionic Megacolon (Hirschsprung Disease), congenital AVM (Arteriovenous Malformation), Congenital Bronchiectasis (Bronchiectasis (Acquired, Congenital)), Congenital Defects (Birth Defects), Congenital distal spinal muscular atrophy, Congenital Dysplastic Angiectasia (Klippel-Trenaunay-Weber Syndrome), Congenital Erythropoietic Porphyria (Porphyria), Congenital facial diplegia, Congenital Glaucoma (Glaucoma), Congenital Heart Disease, Congenital Heart Murmur (Heart Murmur), Congenital Hydrocephalus (Hydrocephalus), Congenital Kyphosis (Kyphosis), Congenital Lymphedema (Lymphedema), Congenital Malformations (Birth Defects), Congenital Methemoglobinemia (Methemoglobinemia), Congenital myasthenic syndrome, Congenital myopathy, Congenital Poikiloderma (Rothmund-Thomson Syndrome), Conjunctivitis (Pink Eye), Connective Tissue Disease, Constipation, Constitutional Hepatic Dysfunction (Gilbert Syndrome), Contact Dermatitis, Contractions, Braxton-Hicks (Braxton Hicks Contractions), Convulsion (Seizure), Cooleys Anemia (Beta Thalassemia), COPD (Chronic Obstructive Pulmonary Disease), Coprolalia (Tourette Syndrome), Corneal Disease, Corneal Ulcer, Coronary Angiogram, Coronary Artery Bypass (Coronary Artery Bypass Graft), Coronary Artery Disease (Heart Disease (Coronary Artery Disease)), Coronary Atherosclerosis, Cortical Dementia (Dementia), Corticobasal Degeneration (Dementia), Costen's Syndrome (Temporomandibular Joint Syndrome (TMJ), Costochondritis and Tietze Syndrome, Coxsackie Virus, CP (Cerebral Palsy), CPAP (Sleep Apnea), CPPD (Pseudogout), Cramp fasciculation syndrome, Cramps, Cranial Arteritis (Polymyalgia Rheumatica), Cranial Dystonia (Dystonia), Craniopharyngioma (Brain Tumor), Craniosynostosis, CRE Infection, CREST Syndrome (Scleroderma), Creutzfeldt-Jakob Disease, crib death (SIDS), Critical Care Pathologies, Crohn Disease (Crohn's Disease), Cryptococcosis, Cryptosporidiosis, CSD (Cat Scratch Disease), CTD (Connective Tissue Disease), CUC (Ulcerative Colitis), Cumulative Trauma Disorder, Curved Spine (Scoliosis), Cushing's Syndrome, Cutaneous lymphoma (Skin cancer), Cutaneous melanoma (Melanoma), Cuts, CVD (Connective Tissue Disease), CVS (Cyclic Vomiting Syndrome (CVS)), Cyclitis (Uveitis), Cyclospora Infection (Cyclosporiasis), Cyclothymic disorder, Cylindrical Bronchiectasis (Bronchiectasis (Acquired, Congenital)), Cystic Fibrosis, Cysticercosis, Cystinuria, Cystitis (Urinary Tract Infection), Cystosarcoma Phyllodes (Breast Cancer), Cytomegalic inclusion body disease, Cytomegalovirus (CMV), Dandruff (Seborrhea), Dandy Fever (Dengue Fever), Dandy-Walker syndrome, Danlos Syndromes (Ehlers-Danlos Syndrome), Dawson disease, De Morsier's syndrome, De Quervain's Tenosynovitis, Deafness, Deep Vein Thrombosis, Degenerative Arthritis (Osteoarthritis), Degenerative Joint Disease (Osteoarthritis), Degenerative Spondylolisthesis (Spondylolisthesis), Deglutition (Swallowing), Dejerine-Klumpke palsy, Dejerine-Sottas disease, Dementia Pugilistica (Dementia), Dementia, Dengue Fever, Depression, Dermabrasion, Dermatitis (Eczema), Dermatomyositis (Polymyositis), Dermatomyositis, Detached Retina (Retinal Detachment), Developmental Coordination Disorder (Learning Disability), Devic's Syndrome, Diabetes Insipidus, Diabetes Mellitus, Diabetes Prevention, Diabetes Treatment, Diabetes, Diabetes, Type 1 (Type 1 Diabetes), Diabetes, Type 2 (Type 2 Diabetes), Diabetic Encephalopathy (Encephalopathy), Diabetic Focal Neuropathy (Diabetic Neuropathy), Diabetic Hyperglycemia (Hyperglycemia), Diabetic Neuropathy, Diarrhea, Difficile, Clostridium (Clostridium Difficile Colitis), Difficulty in Swallowing (Achalasia), Diffuse astrocytoma, Diffuse Fibrosing Alveolitis (Pulmonary Fibrosis), Diffuse Idiopathic Skeletal Hyperostosis, Diffuse sclerosis, Diplopia, Discoid Lupus (Systemic Lupus), Disease Prevention (Prevention), Disease, Behcet's (Behcet's Syndrome), Disease, Carotid Artery (Carotid Artery Disease), Disease, Charcot-Marie-Tooth (Charcot-Marie-Tooth-Disease), Disease, Gaucher (Gaucher Disease), Disease, Graves' (Graves' Disease), Disease, Leigh's (Leigh's Syndrome (Leigh's Disease)), Disease, Marfan (Marfan Syndrome), Disease, Meniere's (Meniere Disease), Disease, Mitochondiral (Mitochondrial Disease), Disease, Non-Polio Enterovirus (Enterovirus (Non-Polio Enterovirus Infection)), Disease, Parkinson's (Parkinson's Disease), Disease, Thyroid (Thyroid Disorders), Disequilibrium of Aging (Vertigo Overview), DISH (Diffuse Idiopathic Skeletal Hyperostosis), Disorder, Asperger (Asperger Syndrome), Disorder, Mitochondrial (Mitochondrial Disease), Disorders of consciousness, Dissociative Identity Disorder, Distal hereditary motor neuropathy type V, Distal Monosomy 1p36 (1p36 Deletion Syndrome), Distal spinal muscular atrophy type 1, Distal spinal muscular atrophy type 2, Disturbed Nocturnal Sleep (Narcolepsy), Diverticular Disease (Diverticulosis), Diverticulitis (Diverticulosis), Diverticulum, Duodenal (Duodenal Diverticulum), Divisum, Pancreas (Pancreas Divisum) Dizziness (Dizzy), DJD (Osteoarthritis), Down syndrome, Dracunculiasis (Guinea Worm Disease), Dravet syndrome, Drug Abuse, Dry Age-Related Macular Degeneration (Macular Degeneration), Dry Eye Syndrome (Dry Eyes), Dry Gangrene (Gangrene), Duchenne muscular dystrophy, Ductal carcinoma, Duodenal Biliary Drainage, Duodenal Diverticulum, Duodenal Ulcer (Peptic Ulcer), DVT (Deep Vein Thrombosis), Dwarfism (Achondroplasia), Dwarfism, Campomelic (Campomelic Dysplasia), Dysarthria, Dysautonomia, Dyscalculia, Dysgeusia (Taste Disorders), Dysgraphia, Dyskinesia, Dyskinetic Cerebral Palsy (Cerebral Palsy), Dyslexia, Dysmenorrhea (Menstrual Cramps), Dysmetabolic Syndrome (Metabolic Syndrome), Dyspepsia, Dysplastic Spondylolisthesis (Spondylolisthesis), Dysthymia, Dystonia, E. Coli, Ear Hematoma (Hematoma), Eating Disorder (Anorexia Nervosa), Eating, Binge (Binge Eating Disorder), Ebola Hemorrhagic Fever (Ebola HF), Echolalia (Tourette Syndrome), Eczema, Edema (Pulmonary), Edema, EDS (Narcolepsy), Ehlers-Danlos Syndrome, EIEC (Enterovirulent E. Coli (EEC)), Eight Day Measles (Measles (Rubeola)), Elephantiasis Congenita Angiomatosa (Klippel-Trenaunay-Weber Syndrome), Elevated Calcium Levels, Elevated Eye Pressure (Glaucoma), Elfin Facies Syndrome (Williams Syndrome), Elfin Facies With Hypercalcemia (Williams Syndrome), Embolism, Pulmonary (Pulmonary Embolism), Emotional Disorders (Mental Health (Psychology)), Emphysema (Lung Condition), Empty sella syndrome, Encephalitis, Encephalocele, Encephalotrigeminal angiomatosis, Encopresis, End Stage Renal Disease (Kidney Failure), Endocarditis, Endometrial Cancer (Uterine Cancer), Endometriosis, End-Stage Renal Disease (Renal Artery Stenosis), Enlarged Organs, Enterovirus (Non-Polio Enterovirus Infection), Enuresis, Environmental Illness (Sick Building Syndrome), Eosinophilic Esophagitis, Eosinophilic Fasciitis, Ependymoma (Brain Tumor), Epidemic Parotitis (Mumps), Epididymitis (Testicular Disorders), Epilepsy (Seizure), Episiotomy, Epithelioid sarcoma, EPO (Erythropoietin), Epstein-Barr Virus (Infectious Mononucleosis), Equilibrium (Vestibular Balance Disorders), Erb's palsy, ERCP, Erythema Infectiosum (Fifth Disease), Erythema Migrans (Lyme Disease), Erythema Nodosum, Erythromelalgia, ESDR (Renal Artery Stenosis), Esophageal Cancer, Esophagitis, Esophagus Cancer, Esophagus Dysplasia (Barrett's Esophagus), Esophagus, Barrett's (Barrett's Esophagus), Ewing Sarcoma (Bone Cancer), Eye cancer, Eye Floaters, Fabry's Disease, Factitious Disorders, Fahr's syndrome, Fainting, Fallopian Tube Removal (Hysterectomy), False Labor (Braxton Hicks Contractions), Familial Hibernation Syndrome (Kleine-Levin Syndrome), Familial Turner Syndrome (Noonan Syndrome), Fatigue, Felty's Syndrome, Fetal Alcohol Syndrome (FAS), Fibrillation (Atrial Fibrillation), Fibrocystic Breast Condition, Fibrocystic Disease of the Pancreas (Cystic Fibrosis), Fibroids (Uterine Fibroids), Fibromyalgia, Fibrosarcoma, Fibrosis, Fibrositis, Fifth Disease, Fire (Burns), Fisher syndrome, Flu (Influenza), Flu, Fluid on the Lungs, Focal Neuropathy, Diabetic (Diabetic Neuropathy), Follicular Carcinoma (Thyroid Cancer), Folliculitis, Folling's Disease (Phenylketonuria), Forestier disease (Diffuse Idiopathic Skeletal Hyperostosis), Fournier's Gangrene (Gangrene), Foville's syndrome, Fracture Hematoma (Hematoma), Fragile X Syndrome, Fragile X-associated tremor/ataxia syndrome, Frambesia (Yaws), Franceschetti-Zwahlen-Klein Syndrome (Treacher Collins Syndrome), Friedreich's ataxia, Frontotemporal Dementia (Dementia), Gallbladder Cancer, Ganser Snydrome (Factitious Disorders), Gardasil HPV Vaccine, Gardner Syndrome, Gastritis, Gastroenteritis (Stomach Flu), Gastrointestinal cancer, Gaucher Disease, Genetic Diseases, German Measles, Gerstmann's syndrome, Giant Platelet Syndrome (Bemard-Soulier Disease), Giardia Lamblia, Gilbert Syndrome, Gingivitis (Gum Disease), Glaucoma, Glioblastoma, Glioma (Brain Tumors), Globoid cell leukodystrophy, Glucocerebrosidase Deficiency (Gaucher Disease), Granulocytopenia (Neutropenia), Granulomatous Vasculitis (Wegener's Granulomatosis), Graves' Disease, Gray matter heterotopia, Guillain-Barré syndrome, Guinea Worm Disease, H. pylori (Helicobacter Pylori), Hair Growth, Hair Loss, Hallervorden-Spatz syndrome, Hamman-Rich Syndrome (Pulmonary Fibrosis), Hansen's Disease (Leprosy), Hantavirus Pulmonary Syndrome, HAPE (Pulmonary Edema), Hashimoto Thyroiditis (Hashimoto's Thyroiditis), Hashimoto's Encephalopathy (Encephalopathy), Hashimoto's Thyroiditis, Hay Fever, HBV Disease (Hepatitis B), HCV Disease (Hepatitis C), HD (Hirschsprung Disease), Head Injury, Headache, Hearing (Deafness), Hearing Loss, Heart Attack, Heart Disease, Heart Failure, Heart Inflammation (Myocarditis), Heart Murmur, Heart Valve Disease, Helicobacter Pylori, Hemangiectatic Hypertrophy (Klippel-Trenaunay-Weber Syndrome), Hemangioendothelioma, Hemangioma, Hepatic (Hepatic Hemangioma), Hematoma, Hemiplegia, Hemolytic Uremic Syndrome, Hemorrhage, Henoch-Schonlein Purpura, Hepatic Dysfunction, Constitutional (Gilbert Syndrome), Hepatic Hemangioma, Hepatitis (Viral Hepatitis), Hepatocellular Carcinoma, Hereditary Coproporphyria (Porphyria), Hereditary motor neuropathies, Hereditary Pancreatitis (Pancreatitis), Hereditary spastic paraplegia, Heredopathia atactica polyneuritiformis, Heritable Disease (Genetic Disease), High Blood Pressure, Hirayama syndrome, Hirschsprung Disease, Hodgkin lymphoma, Hodgkins Disease, Holmes-Adie syndrome, Holoprosencephaly, HSP (Henoch-Schonlein Purpura), Hughes Syndrome (Antiphospholipid Syndrome), Human Immunodeficiency Virus (HIV), Huntington's disease, HUS (Hemolytic Uremic Syndrome), Hutchinson-Gilford Syndrome (Progeria Syndrome), Hydranencephaly, Hydrocephalus, Hydronephrosis, Hydrophobia (Rabies Virus), Hypercalcemia-Supravalvar Aortic Stenosis (Williams Syndrome), Hypercortisolism (Cushing's Syndrome), Hyperglycemia, Hyperhidrosis, Hyperkalemia, Hyperkinetic Impulse Disorder, Hypermobility Syndrome, Hypernephroma (Kidney Cancer), Hyperparathyroidism, Hyperplasia, Hypertension, Hyperthyroidism, Hypertrophic Cardiomyopathy, Hypertrophy, Hypogeusia (Taste Disorders), Hypoglycemia, Hyponatremia, Hypoparathyroidism, Hypopharyngeal cancer, Hypotension (Low Blood Pressure), Hypotensive Encephalopathy (Encephalopathy), Hypothalamic Disease (Hypothyroidism), Hypothyroidism, Hypotonic Cerebral Palsy (Cerebral Palsy), Hypoxia, IBS (Irritable Bowel Syndrome (IBS)), Idiopathic Endolymphatic Hydrops (Meniere Disease), Idiopathic Intracranial Hypertension (Pseudotumor Cerebri), Idiopathic Pulmonary Fibrosis (Pulmonary Fibrosis), Idiopathic Pulmonary Hypertension (Pulmonary Hypertension), Idiopathic Thrombocytopenic Purpura (ITP), Ileitis (Crohn's Disease), Ileocolitis (Crohn's Disease), Iliotibial Band Syndrome, Immunological Diseases, Impingement Syndrome, Inclusion body myositis, Increased-Permeability Pulmonary Edema (ARDS), Infantile Acquired Aphasia (Landau-Kleffner Syndrome), Infections, Inflammatory Breast Cancer, Inflammatory Diseases, Inflammed Organs, Influenza, Injuries, Inner Ear Trauma, IPF (Pulmonary Fibrosis), Iritis, Irritable Bowel Syndrome (IBS), Isaac's Syndrome, Ischemia Reperfusion Injury, Ischemic Colitis (Colitis), Ischemic Heart Disease, Ischemic Nephropathy (Renal Artery Stenosis), Ischemic Priapism, Ischemic Renal Disease (Renal Artery Stenosis), Ischial Bursitis (HipBursitis), Isthmic Spondylolisthesis (Spondylolisthesis), IT Band Syndrome (Iliotibial Band Syndrome), Jakob-Creutzfeldt Disease (Creutzfeldt-Jakob Disease), Jaundice, Joint Hypermobility Syndrome (Hypermobility Syndrome), Joubert syndrome, Karak syndrome, Kawasaki Disease, Kearns-Sayre syndrome, Keratitis, Keratoconus, Keratosis Pilaris, Kernicterus, Kidney cancer, Kidney Disease, Kidney Dysplasia, Kidney Failure, Kinsbourne syndrome, Kleine-Levin syndrome, Klinefelter Syndrome, Klippel Feil syndrome, Klippel Trenaunay-Weber Syndrome, KLS (Kleine-Levin Syndrome), Knee Bursitis, Krabbe disease, KTS (Klippel-Trenaunay-Weber Syndrome), Kufor-Rakeb syndrome, Lafora disease, Lambert-Eaton myasthenic syndrome, Lambliasis (Giardia Lamblia), Landau-Kleffner syndrome, Laryngeal Carcinoma (Larynx Cancer), Laryngitis, Larynx Cancer, Lateral Femoral Cutaneous Nerve Syndrome (Meralgia Paresthetica), Lateral medullary (Wallenberg) syndrome, Lattice Dystrophy (Corneal Disease), Learning Disabilities, Legionnaire Disease, Legs, Restless (Restless Leg Syndrome), Leigh's disease, Leiomyosarcoma (Soft tissue sarcoma), Leishmaniasis, Lennox-Gastaut syndrome, Leprosy, Lesch-Nyhan syndrome, Lesions, Leukemia, Leukodystrophy, Leukopathia (Vitiligo), Leukopenia (Neutropenia), Leukoplakia, Lewy Body Dementia (Dementia), Lichen Planus, Lichen Sclerosus, Linear Scleroderma (Scleroderma), Lipoid histiocytosis (Gaucher Disease), Liposarcoma, Listeria, Liver cancer, Liver Cirrhosis (Cirrhosis), Liver Disease, LKS (Landau-Kleffner Syndrome), Lobe Resection (Surgical Options for Epilepsy), Lobular carcinoma, Loeys-Dietz Syndrome, Lou Gehrig's Disease (Amyotrophic Lateral Sclerosis), Low Energy Lumbar Stenosis, Lung Cancer, Lyme Disease, Lymph Node (Swollen Lymph Nodes), Lymphapheresis (Hemapheresis), Lymphedema, Lymphoblastic Leukemia, Lymphocytic Colitis, Lymphocytic Thyroiditis (Hypothyroidism), Lymphoma, Machado-Joseph disease, Macrencephaly, Macular Degeneration, Mad Cow Disease, Mal de debarquement, Malaria, Malignancy (Cancer), Marfan Syndrome, Martin-Bell Syndrome (Fragile X Syndrome), Measles, Medullary Carcinoma, Medulloblastoma (Brain Tumor), Megacolon (Hirschsprung Disease), Melanoma, MELAS Syndrome, Melasma, Melioidosis, Melkersson Rosenthal syndrome, Memory Loss (Dementia), Meniere Disease, Meningioma (Brain Tumor), Meningitis, Meningococcemia, Menkes disease, Mental Health (Psychology), Mental Illness, Mesothelioma, Metabolic Encephalopathy (Encephalopathy), Metabolic Syndrome, Metachromatic leukodystrophy, Metastasized Cancer, Methicillin Resistant Staphylococcus Aureus (MRSA Infection), Microcephaly, Migraines, Mitochondrial Diseases, Mitochondrial Disorders, Mitochondrial Dysfunction, Mitochondrial myopathy, Mobius syndrome, Morphea (Scleroderma), Morton's Neuroma, Morvan syndrome, Motor skills disorder, Mountain Sickness, Movement Disorders, Moyamoya disease, Mucocutaneous Lymph Node Syndrome (Kawasaki Disease), Multiple Myeloma, Multiple Organ Failure, Multiple Personality Disorder (Dissociative Identity Disorder), Multiple Sclerosis (MS), Munchausen Syndrome, Muscular dystrophy, Musculoskeletal Pain, Myalgic Encephalomyelitis (Chronic Fatigue Syndrome), Myasthenia Gravis Myelodysplastic Syndrome, Myeloma (Multiple Myeloma), Myopathy, Myotonia congenita, Myotonic dystrophy, Myotubular myopathy, Narcolepsy, Nasopharyngeal Cancer, Neoplasm (Cancer), Nerve Disorders, Neuro-Behget's disease, Neuroblastoma, Neurodermatitis (Atopic Dermatitis), Neurofibromatosis, Neuroleptic malignant syndrome, Neurological Disorders, Neuromuscular Disorders, Neuropathy, Neurosis, Neutropenia, Niemann-Pick disease, NISP (Pulmonary Fibrosis), NMO (Devic's Syndrome), Nodules, Non-Hodgkin Lymphoma, Non-Hodgkin's Lymphoma, Non-small cell lung cancer (NSCLC), Noonan Syndrome, Norovirus (Norovirus Infection), Obesity, Obsessive Compulsive Disorder (OCD), Ocular cancer, Ohtahara syndrome, Oligodendroglioma (Brain Tumor), Olivopontocerebellar atrophy, Open Angle Glaucoma (Glaucoma), Opsoclonus myoclonus syndrome, Optic Neuritis, Optic Neuropathy (Glaucoma), Oral Cancer, Oral Health Problems, Organ Diseases and Dysfunctions, Organ Failure, Organ Transplantation, Osgood-Schlatter Disease, Osteitis Deformans (Paget's Disease), Osteoarthritis, Osteochondritis Dissecans, Osteodystrophy, Osteogenic sarcoma (Bone cancer), Osteohypertrophic Nevus Flammeus (Klippel-Trenaunay-Weber Syndrome), Osteomalacia (Osteodystrophy), Osteomyelitis, Osteonecrosis (Aseptic Necrosis), Osteopenia, Osteoporosis, Osteosarcoma, Ovarian Cancer, Overuse Syndrome (Repetitive Motion Disorders (RMDs)), Oxidative Stress Disorders, Paget's Disease, Pain, Pancreas Cancer (Pancreatic Cancer), Pancreas Fibrocystic Disease (Cystic Fibrosis), Pancreatitis, PANDAS, Panic Attack (Panic Disorder), Panniculitis, Idiopathic Nodular (Weber-Christian Disease), Panuveitis (Uveitis), Papillary Carcinoma, Paralysis, Paranasal sinus cancer, Paraneoplastic diseases, Parkinson's Disease, Parry-Romberg Syndrome, Patellofemoral Syndrome, Pelizaeus-Merzbacher disease, Pendred Syndrome, Pericarditis, Pericoronitis, Perilymphatic Fistula (Vestibular Balance Disorders), Peripheral Vascular Disease, Pervasive Development Disorders, Pick's disease, Pinched nerve, Plantar Fasciitis (Heel Spurs), PMDD (Premenstrual Dysphoric Disorder (PMDD)), Pneumonia, Poisoning, Polio, Polycystic Kidney Disease, Polycystic Ovary, Polycystic Renal Disease (Polycystic Kidney Disease), Pontiac Fever, Post-Myocardial Infarction, Postoperative Pancreatitis (Pancreatitis), Postpartum Depression, Post-stroke, Post-Surgery, Posttraumatic Stress Disorder, Prader Willi Syndrome (Prader-Willi Syndrome), Preexcitation Syndrome (Wolff-Parkinson-White Syndrome), Premenstrual Dysphoric Disorder (PMDD), Primary central nervous system (CNS) lymphoma, Primary Dementia (Dementia), Primary lateral sclerosis, Primary Lymphedema (Lymphedema), Progeria Syndrome, Progressive Systemic Sclerosis (Scleroderma), Prostate cancer, Psoriasis, Psoriatic Arthritis, Psychiatric Disorders, Psychological Disorders, Psychosis (Schizophrenia), PTSD (Posttraumatic Stress Disorder), Pubertal Gynecomastia (Gynecomastia), Pulmonary Fibrosis, Reflex neurovascular dystrophy, Renal cell carcinoma, Renal Disease (Renal Artery Stenosis), Renal Failure (Kidney Failure), Reperfusion Injury, Restless Leg Syndrome, Retinoblastoma, Rheumatoid Arthritis, Rosacea, Rubella (German Measles), Salivary Gland Cancer, Sarcoma, Schizophrenia, Seborrheic Dermatitis, Secondary Glaucoma (Glaucoma), Seizures, Sepsis, Septic Arthritis, Septic Shock, Septicemia (Sepsis), Sick Building Syndrome, Sinusitis, Skin cancer, Skin Conditions, Skin Diseases, Sleep Apnea, Small cell lung cancer (SCLC), Soft tissue sarcoma, Spina Bifida, Spinal and bulbar muscular atrophy, Spinal cancer, Spinal Cord Injury, Spinal muscular atrophy, Spinal Stenosis (Lumbar Stenosis), Spine Curvature (Scoliosis), Squamous Cell Carcinoma, Stomach Cancer, Stomach Cancer, Stress, Stroke Prevention, Stroke Treatment, Stroke, Subacute Thyroiditis (Thyroiditis), Subclinical Hypothyroidism (Hypothyroidism), Subconjunctival Hemorrhage, Subcortical Arteriosclerotic Encephalopathy (Binswanger's Disease), Surgery, Syndrome, Asperger (Asperger Syndrome), Syndrome, Behcet's (Behcet's Syndrome), Systemic Lupus, Takayasu Disease, Tarsal Tunnel Syndrome (Carpal Tunnel Syndrome), Tay-Sachs disease, TB (Tuberculosis (TB)), Teeth Conditions, Tendinitis, Testicular Cancer, Testicular Disorders, Tetanus, Throat cancer, Thyroid Cancer, Thyroid Disorders, Tourette Syndrome, Toxic Shock Syndrome (TSS), Transient ischemic attack, Transplant, Heart (Heart Transplant), Trauma (Posttraumatic Stress Disorder), Traumatic brain injury, Tremors, Trouble Breathing, Tuberculosis (TB), Tumors, Type 2 Diabetes, Ulcerative Colitis, Uterine Cancer, Vascular Diseases, Vertigo, Viral Infections, Williams Syndrome, Wilson's disease, or Zellweger syndrome.

### Crossing the Blood-Brain Barrier

Cationic and other charged molecules have a very difficult time crossing the blood brain barrier (BBB). As a result, methods are required that allow drugs to cross the BBB and stay there. In one method, a chemical drug delivery system (CDS), utilizes enzymes to activate prodrugs and give molecules a cationic charge once they cross into tissues from blood vessels (Bodor and Buchwald, 2002).

The diagram above provides a mechanism for this system utilizing the reactive lipid and MPTP-targeting moieties discussed previously in this application. The compound (1) comprises a 1,4-dihydro-N-methylnicotinic acid (dihydrotrigonelline) prodrug moiety on the left side, and a reactive dienol ether. Upon reaction with a reactive species, this compound produces vitamin B3 and MPTP-binding phenylglyoxal. This lipophilic prodrug can cross into tissues, including the central nervous system (CNS). Upon entry into the CNS, the dihydrotrigonelline moiety is deprotonated by NADH dehydrogenase (which is depleted in blood), converting it into an activated trigonelline structure. This activated structure now has a charged cationic pyridinium group, which does not allow it to re-cross the blood brain barrier and enter back into the rest of the body. Therefore, any prodrugs of this general design that enters the central nervous system (CNS) and were activated by this enzyme, is now retained inside the CNS (“locked-in”), while the activated drugs in the rest of the body will be excreted efficiently. This allows for an accumulation in therapeutic quantities of drugs in the CNS. Even better, the ester group on the trigonelline moiety prodrug is slowly hydrolyzed over days and weeks, giving a steady supply of activated drugs to the CNS tissues, while the prodrug moiety—trigonelline—is naturally metabolized into niacin, or vitamin B3, so there is no accumulation of positive charges or unwanted byproducts from this reaction that remain in the CNS or brain.

The cationic group on the pyridinium moiety is particularly useful for targeting the mitochondria, and particularly the inner mitochondrial membrane (IMM) because cationic charges accumulate rapidly into the mitochondria. If they have both a cationic charge, along with a lipophilic chain or group, they are inserted into the IMM membrane bilayer, on both the outer and inner leaflets, where cardiolipin and other critical lipids are located.

Thus, in some embodiments, a nonpolar compound capable of crossing the blood-brain barrier (BBB) into the central nervous system (CNS) is provided. In these embodiments, the compound reacts with an activating agent in the CNS to form an active cationic product that reacts with a mitochondrial reactive lipid species and/or inhibits or enhances the opening of a mitochondrial permeability transition pore (MPTP).

The activating agent in these embodiments can be any enzyme that is more highly expressed in the CNS than in the bloodstream. Alternatively, if the compound is administered directly into the CNS, the enzyme need only be any enzyme that is expressed in the CNS, without concern for whether it is differentially expressed in the CNS. Non-limiting examples of such enzymes include carboxylesterase, acetylcholinesterase, esterase, butyrylcholinesterase, paraoxonase, matrix metalloproteinase, alkaline phosphatase, β-glucuronidase, human valacyclovirase, hydrolase, plasmin, protease, prostate-specific antigen, purine-nucleoside phosphorylase, carboxypeptidase G2, penicillin amidase, β-lactamase, β-galactosidase, cytosine deaminase, serine hydrolase, cholinesterase, phosphorylase, acetaldehyde dehydrogenase, dehydrogenase, aldehyde oxidase, amino acid oxidase, oxidase, P450 Reductase, DT-diaphorase, thymidine phosphorylase, glutathione S-transferase, deoxycytidine kinase, lyase, thymidylate synthase, tyrosinase, methionine δ-lyase, cytosine deaminase, β-lactamase, penicillin amidase, carboxypeptidase, β-glucuronidase, thymidine kinase, and nitroreductase. In various embodiments, the activating agent is NADH dehydrogenase.

The activation of a generic compound that targets mitochondrial reactive lipid species by NADH dehydrogenase in the CNS is shown above.

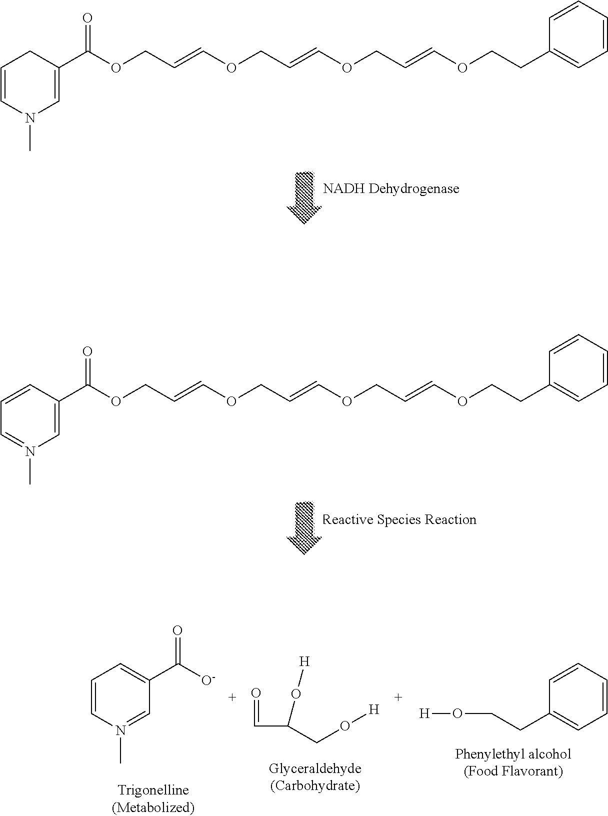

In the above diagram, the top two compounds are both useful. The top compound can cross the BBB, owing to its lipophilic character. The second compound cannot cross the BBB due to its positive charge, and will target the inner mitochondrial matrix (IMM) for that same reason. The four vinyl ether moieties increase the compound's affinity for the IMM. This compound will not directly affect the MPTP; its products are all non-toxic.

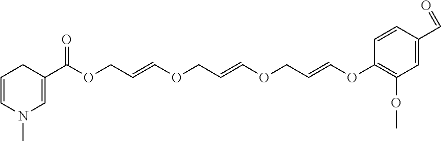 [SMILES: COc1cc(C=O)ccc1O/C=C/CO/C=C/CO/C=C/COC(=O)C1=CN(C)C=CC1]

The prodrug depicted above also targets lipid peroxides but not MPTP since it forms vanillin and glyceraldehyde upon reaction with reactive lipid species.

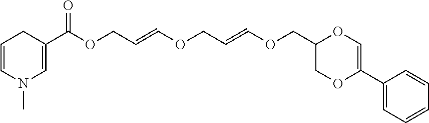 [SMILES: CN1C=CCC(C(=O)OC/C=C/OC/C=C/OCC2COC(c3ccccc3)=CO2)=C1]

The molecule above is a prodrug that will cross the BBB and skin, and the activated version shown below is designed to insert itself into the IMM. There are three reactive lipid species and reactive species sensitive groups to it, which will allow it to reach far into the IMM, and upon reaction, form glycerol, phenylglyoxal (MPTP inhibitor), glyceraldehyde, and trigonelline. This compound thus targets and inhibits MPTP opening, and targets cardiolipin hydroperoxides and

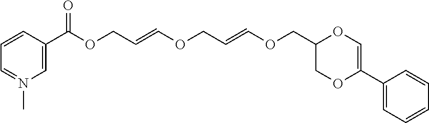

cardiolipin peroxyl radicals.

The above compound is the activated molecule from the one shown above, and can inserted like this directly into a person or topically to treat the skin, or be enzymatically activated once the prodrug version at the top passes from the bloodstream and into the tissues.

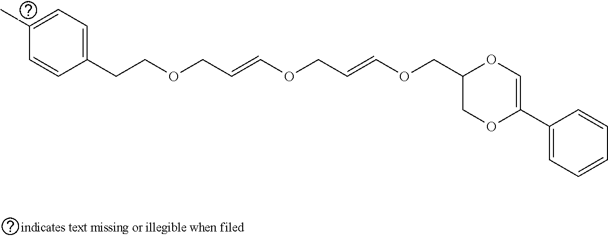 [SMILES: Cc1ccc(CCOC/C=C/OC/C=C/OCC2COC(c3ccccc3)=CO2)cc1]

By contrast with previous compounds, the above compound does not using the trigonelline prodrug moiety, but has permanent cationic charge. This compound can be administered intravenously, or used topically to treat the skin, and would not cross the BBB.

Consistent with the example immediately above, permanent and preformed charges on prodrugs do not cross the BBB, which can be important if you only want to treat mitochondrial dysfunction in non-nervous tissue, or if fast treatment and protection is desired, such as after a myocardial infarction, since the enzymatic conversion required for other compounds is not required.

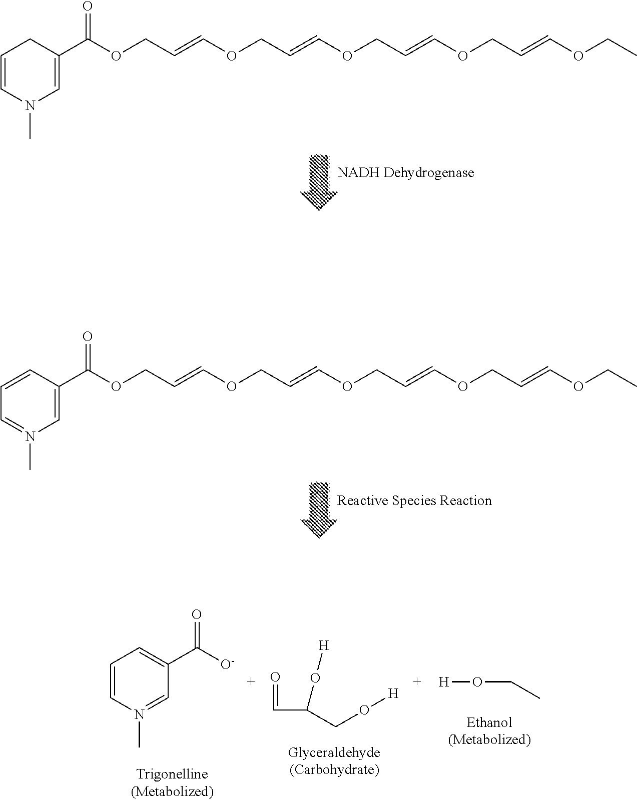

The diagram above shows a long chain prodrug that is designed to be soluble in water, but highly lipophilic, so it crosses the BBB, activated in nervous tissue, where the activated compound accumulates in the brain, targeting the IMM, and has more than one vinyl ether along its chain that allows for a high activity against hydroperoxide lipids and peroxyl lipids, specifically cardiolipin. Upon reaction with reactive species, the compound yields glyceraldehyde, trigonelline, and ethanol products, all of which are metabolized. The compound can be easily modified such that an alcohol different from ethanol is produced. The MPTP is not targeted since α-dicarbonyl groups are not produced.

The compound below is similar to the above compound, except the compound yields a food flavorant product rather than ethanol.

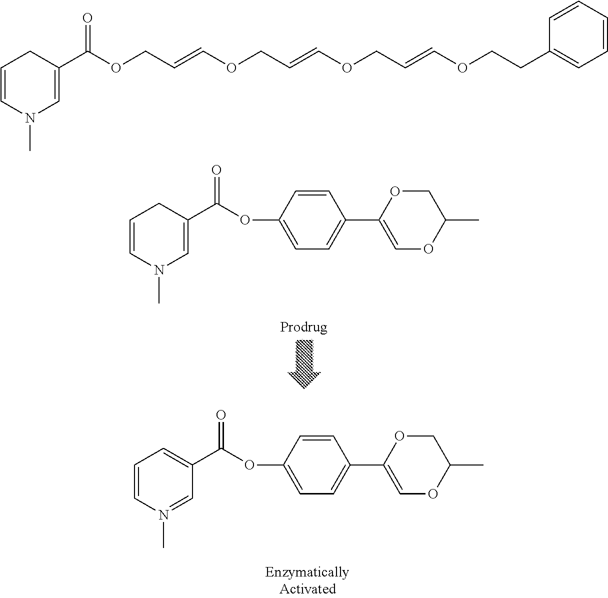

The molecule and mechanism above produces 4-hydroxyphenyl glyoxal after both reactive species and enzymatic activation. 4-hydroxyphenyl promotes the opening of the MPTP, causing mitochondrial dysfunction, useful for treating diseases such as cancer where cellular apoptosis is desired. Because the top compound can cross the BBB, this compound is useful for treating cancers of the nervous system, such as brain cancers.

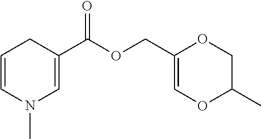 [SMILES: CC1COC(COC(=O)C2=CN(C)C=CC2)=CO1]

The prodrug above will also activate the MPTP, but since the α-dicarbonyl is not conjugated, which helps make the arginine binding irreversible, this is useful as a reversible MPTP opening molecule designed for treating brain tumors upon both chemical and enzymatic activation.

The panel below provides compounds to prevent compounds targeted for the central nervous system (CNS) from affecting mitochondria in non-CNS locations

| | |
|---|---|
| 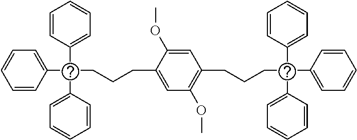 [SMILES: COc1cc(CCCC(c2ccccc2)(c2ccccc2)c2ccccc2)c(OC)cc1CCCC(c1ccccc1)(c1ccccc1)c1ccccc1] | This molecule administered prior to delivering our prodrug chemo medications will depolarize mitochondrial membrane potentials everywhere in the body ~ except the brain and CNS since it is way too massive to cross the blood brain barrier This technology could be perfect for allowing our prodrugs to just target brain cancer and other metastasized tumors in the CNS, while leaving the rest of the body and organs alone and unharmed |
| | This is because our delocalized [symbol] cationic |
| | prodrugs will be attracted to the negatively charged |
| | mitochondria in the body, so only the brain cells' |
| | mitochondria will still be hyperpolarized and will |
| | be highly attracted towards these prodrugs |
| | As a result, we should be able to administer higher |
| | dosages of our prodrugs after an injection of |
| | these adjuvant molecules, since there will be less |
| | toxicity concerns in other organs, so this |
| | combined chemical approach can help yield better |
| | results for conditions such as brain cancer |
| 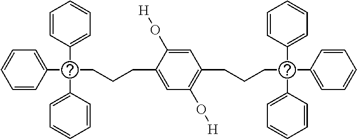 [SMILES: Oc1cc(CCCC(c2ccccc2)(c2ccccc2)c2ccccc2)c(O)cc1CCCC(c1ccccc1)(c1ccccc1)c1ccccc1] | Besides having the same properties as the molecule above, this particular structure is also an antioxidant, specifically the phenol aspect in the middle This will allow us to not only depolarize the cells in non-CNS cells and non-CNS parts of the body, but also help prevent any prodrugs from being activated, that might happen to wander into a non-CNS mitochondria By removing the ROS, specifically ROS that are |
| | close to the inner mitrochondrial membrane on the |
| | matrix side ~ where both our prodrugs and these |
| | antioxidants will co-occupy the space together, we |
| | can react with ROS and remove them before they |
| | activate our prodrugs |
| | This molecule provides two methods for giving |
| | CNS-specificity for our prodrugs |
| 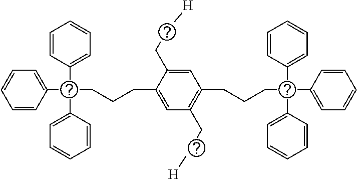 [SMILES: CCc1cc(CCCC(c2ccccc2)(c2ccccc2)c2ccccc2)c(CC)cc1CCCC(c1ccccc1)(c1ccccc1)c1ccccc1] | Besides having the same depolarizing properties for non-CNS cellular mitochondria as the two molecules above, this molecule will specifically react with prodrugs after they have formed in the mitochondrial matrix This molecule will prevent the activated prodrugs from reaching the MPTP as they travel along the mitochondrial matrix membrane because the thiol goups love to attack aldehydes, just like the thiol found in [symbol] All three of these structures also have a chain-link |
| | fence property to them as well, where there will be |
| | a physical horizontal blockage on the membrane |
| | preventing activated chemo medications from |
| | traveling along the matrix membrane and reaching |
| | their MPTP targets |

[symbol]

indicates data missing or illegible when filed

Also provided herewith is any of the compounds discussed above formulated in a pharmaceutical excipient. Such pharmaceutical preparations are extensively discussed at pp. 22-26 of WO 2017/049305 and pp. 53-59 of WO 2018/102463, both sections incorporated by reference.

### REFERENCES

Anderson E J, Katunga L A, Willis M S. Mitochondria as a Source and Target of Lipid Peroxidation Products in Healthy and Diseased Heart. Clinical and experimental pharmacology & physiology. 2012; 39(2):10.1111/j.1440-1681.2011.05641.x. doi:10.1111/j.1440-1681.2011.05641.x.
N. Bodor and P. Buchwald, “Barriers to Remember: Brain-Targeting Chemical Delivery Systems and Alzheimer's Disease,” Drug Discovery Today, 7(14), 766-774 (2002).
Braverman N. E., Moser, A. B. (2012) Functions of plasmalogen lipids in health and disease, Biochim. Biophys. Acta, 1822:1442-1452.
Bhutia et al. (2016) Biochem. J. 473:1503-1506.
Eirin et al. (2017) Handb. Exp. Pharmacol. 240:229-250.
Eriksson, O., Fontaine, E., and Bernardi, P. (1998) Chemical modification of arginines by2,3-butanedione and phenylglyoxal causes closure of the Mitochondrial Permeability Transition Pore. J. Biol. Chem. 273: 12669-12674.
Frantz, M-C. and Wipf, P. (2010) Environ. Mol. Mutagen. 51:462-475.
Garciá-Ayllón M-S, Small D H, Avila J, Sáez-Valero J. (2011) Revisiting the Role of Acetylcholinesterase in Alzheimer's Disease: Cross-Talk with P-tau and β-Amyloid. Frontiers in Molecular Neuroscience. 4: Article 22, 9 pages.
Görlach, A., Bertram, K., Hudecova, S., & Krizanova, O. (2015). Calcium and ROS: A mutual interplay. Redox Biology, 6, 260-271.
Guo C, Sun L, Chen X, Zhang D. Oxidative stress, mitochondrial damage and neurodegenerative diseases. Neural Regeneration Research. 2013; 8(21):2003-2014. doi:10.3969/j.issn.1673-5374.2013.21.009.
Hossion A M L, Bio M, Nkepang G, Awuah S G, You Y. (2013) Visible Light Controlled Release of Anticancer Drug through Double Activation of Prodrug. ACS Medicinal Chemistry Letters. 4:124-127.
Hurst et al. (2017) J Bioenerg Biomembr. 49: 27-47.
Javadov et al. (2017) Cell Mol Life Sci. 74: 2795-2813.
Johans, M., Milanesi, E., Frank, M. et al. (2005) Modification of permeability transition pore arginine(s) by phenylglyoxal derivatives in isolated mitochondria and mammalian cells. Structure-function relationship of arginine ligands, J. Biol. Chem. 280:12130-12136.
Kristal, Bruce & K Park, B & Yu, Byung. (1996). 4-Hydroxyhexenal Is a Potent Inducer of the Mitochondrial Permeability Transition. The Journal of biological chemistry. 271. 6033-8. 10.1074/jbc.271.11.6033.
Kumar A, Singh A. (2015) A review on mitochondrial restorative mechanism of antioxidants in Alzheimer's disease and other neurological conditions. Frontiers in Pharmacology 6:206.
Linder, M. D.; Morkunaite-Haimi, S.; Kinnunen, P. K. J.; Bernardi, P.; Eriksson, O. (2001) Ligand-selective modulation of the permeability transition pore by arginine modification. Opposing effects of p-hydroxyphenylglyoxal and phenylglyoxal. Journal of Biological Chemistry 277(2): 937-942.
Maleth, M. and Hegyi, P. (2016) Phil. Trans. R. Soc. B 371: 20150425.
Motloch et al. (2015) Oxidative Medicine and Cellular Longevity, Article 234104, 8 pages.
Murphy, M. P. (2008) Targeting lipophilic cations to mitochondria. Biochim. Biophys. Acta 1777: 1028-1031.
Nakagawa Y. (2004) Initiation of Apoptotic Signal by the Peroxidation of Cardiolipin of Mitochondria. In: Lee H. K., DiMauro S., Tanaka M., Wei Y H. (eds) Mitochondrial Pathogenesis. Annals of the New York Academy of Sciences, vol 1011. Springer, Berlin, Heidelberg.
Petrosillo, G., M. Matera, G. Casanova, F. M. Ruggiero, G. Paradies, Mitochondrial dysfunction in rat brain with aging: Involvement of complex I, reactive oxygen species and cardiolipin, Neurochemistry International, Volume 53, Issue 5, 2008, Pages 126-131.
Ponnalagu, D. and Singh, H. (2017) Handb Exp Pharmacol. 2017; 240: 71-101.
Prentice et al. (2015) Oxidative Medicine and Cellular Longevity, Article 964518, 7 pages.
Rajaputra P, Bio M, Nkepang G, Thapa P, Woo S, You Y. (2016) Anticancer Drug Released from Near I R-activated Prodrug Overcomes Spatiotemporal Limits of Singlet Oxygen. Bioorganic & medicinal chemistry 24:1540-1549.
Rao, V. K., Carlson E. A., Yan, S. S. (2014) Mitochondrial permeability transition pore is a potential drug target for neurodegeneration, Biochimica et Biophysica Acta (BBA)—Molecular Basis of Disease, 1842, 8, 1267
Rooseboom et al. (2004) Pharmacol. Rev. 56:53-102.
Ross, M F, T. Da Ros, F. H. Blaikie, T. A. Prime, C. M. Porteous, I. I. Severina, V. P. Skulachev, H. G. Kjaergaard, R. A. Smith, M. P. Murphy, (2006) Accumulation of lipophilic dications by mitochondria and cells, Biochem. J. 400:199-208.
Rottenberg, H. and Joek, J. (2017) Aging Cell 16:943-955.
Severina I I, Vyssokikh M Y, Pustovidko A V, Simonyan R A, Rokitskaya T I, and Skulachev V P (2007) Effects of lipophilic dications on planar bilayer phospholipid membrane and mitochondria. Biochim Biophys Acta 1767:1164-1168.
Sheu, S. S., Nauduri, D. and Anders, M. W. (2006). Targeting antioxidants to mitochondria: A new therapeutic direction. Biochim Biophys Acta, 1762:256-265.
Sileikyte et al. (2015) Small Molecules Targeting the Mitochondrial Permeability Transition. Probe Reports from the NIH Molecular Libraries Program [Internet].
Snow B J, Rolfe F L, Lockhart M M, Frampton C M, O'Sullivan J D, Fung V, Smith R A, Murphy M P, Taylor K M (2010) A double-blind, placebo-controlled study to assess the mitochondria-targeted antioxidant MitoQ as a disease-modifying therapy in Parkinson's disease. Mov Disord 25(11):1670-1674.
Speer O, Morkunaite-Haimi S, Liobikas J, Franck M, Hensbo L, Linder M D, Kinnunen P K, Wallimann T, Eriksson O. (2003) Rapid suppression of mitochondrial permeability transition by methylglyoxal. Role of reversible arginine modification. J Biol Chem. 278:34757-34763.
Stadelmann-Ingrand S., Favreliere S., Fauconneau B., Mauco G., and Tallineau C. (2001) Plasmalogen degradation by oxidative stress: Production and disappearance of specific fatty aldehydes and fatty α-hydroxyaldehydes. Free Radical Biol. Med. 31:1263-1271.
Sun et al. (2014) BioMed Res. Int., Article 805926, 9 pages.
Wong J R, Ho C, Mauch P, Coleman N, Berman S, Chen L B (1995) Removal of carcinoma cells from contaminated bone marrow using the lipophilic cation rhodamine 123. Clin. Cancer Research 1:621-630.
Sun et al. (2014) BioMed Res. Int., Article 805926, 9 pages.
Wagner, G. and Payne, R. (2011) J. Aging Res., Article 234875, 12 pages.
Wongrakpanich et al. (2014) Nanomedicine (Lond.) 9:2531-2543.
Ye, X.-Q., Li, Q., Wang, G.-H., Sun, F.-F., Huang, G.-J., Bian, X.-W., Yu, S.-C. and Qian, G.-S. (2011) Mitochondrial and energy metabolism-related properties as novel indicators of lung cancer stem cells. Int. J. Cancer, 129: 820-831. doi:10.1002/ijc.25944.
Zhang J, Wang X, Vikash V, et al. ROS and ROS-Mediated Cellular Signaling. Oxidative Medicine and Cellular Longevity. 2016; 2016:4350965. doi:10.1155/2016/4350965.
Zorov D B, Juhaszova M, Sollott S J. Mitochondrial Reactive Oxygen Species (ROS) and ROS-Induced ROS Release. Physiological Reviews. 2014; 94(3):909-950. doi:10.1152/physrev.00026.2013.
PCT Patent Publication WO 2016/023015.
PCT Patent Publication WO 2017/049305.
PCT Patent Publication WO 2018/102463.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A compound that reacts with a reactive lipid species to form a non-toxic product or an active product, wherein the compound comprises any of the following structures: 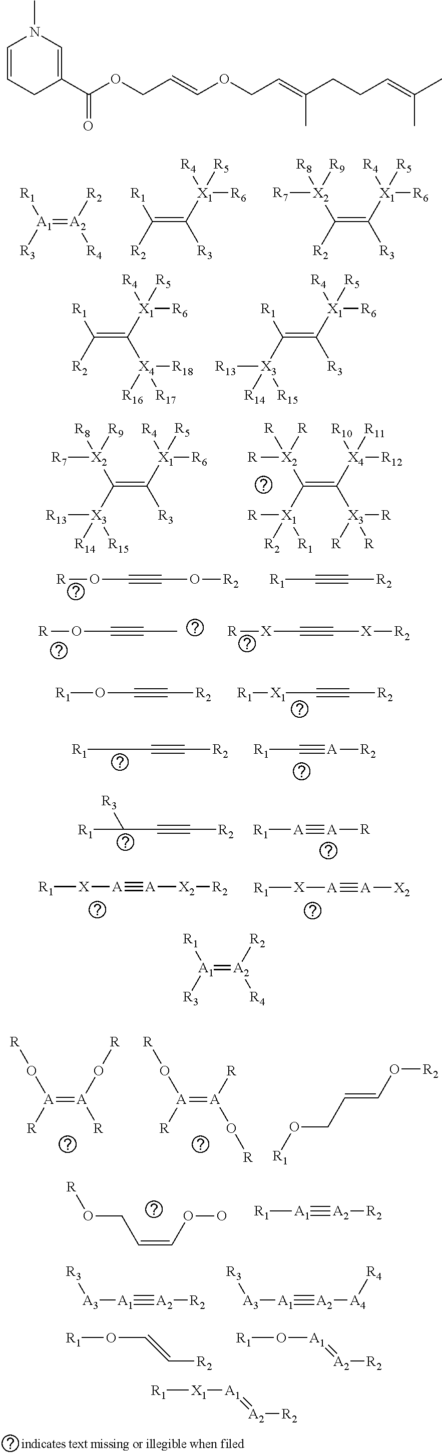
wherein
A₁, A₂, A₃ and A₄ are each independently C, S, N, Si or P;
X₁, X₂, X₃ and X₄ are each independently O, N, S, or P; and
R₁-R₁₈ are each independently a lone pair of electrons, hydrogen, a substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, an electron-donating group (e.g., O, N, P, or S), electron-withdrawing group, a halogen, a resonating or non-resonating moiety, a conjugated or non-conjugated moiety, substituted or unsubstituted arylalkyl, or substituted or unsubstituted heteroarylalkyl, wherein where more than one R groups are present, two R groups can join together to form a ring.

2. The compound of claim 1, comprising the structure

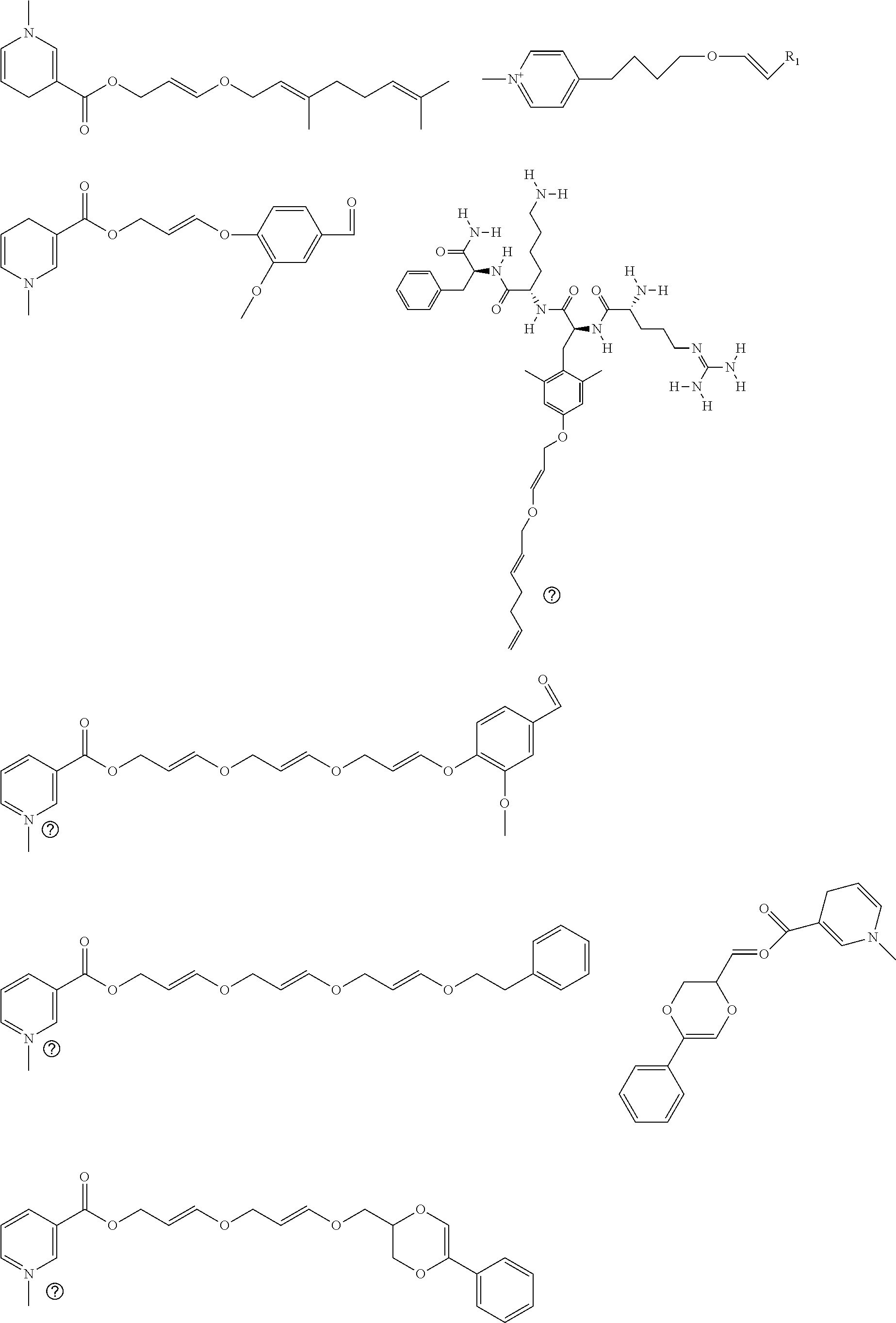

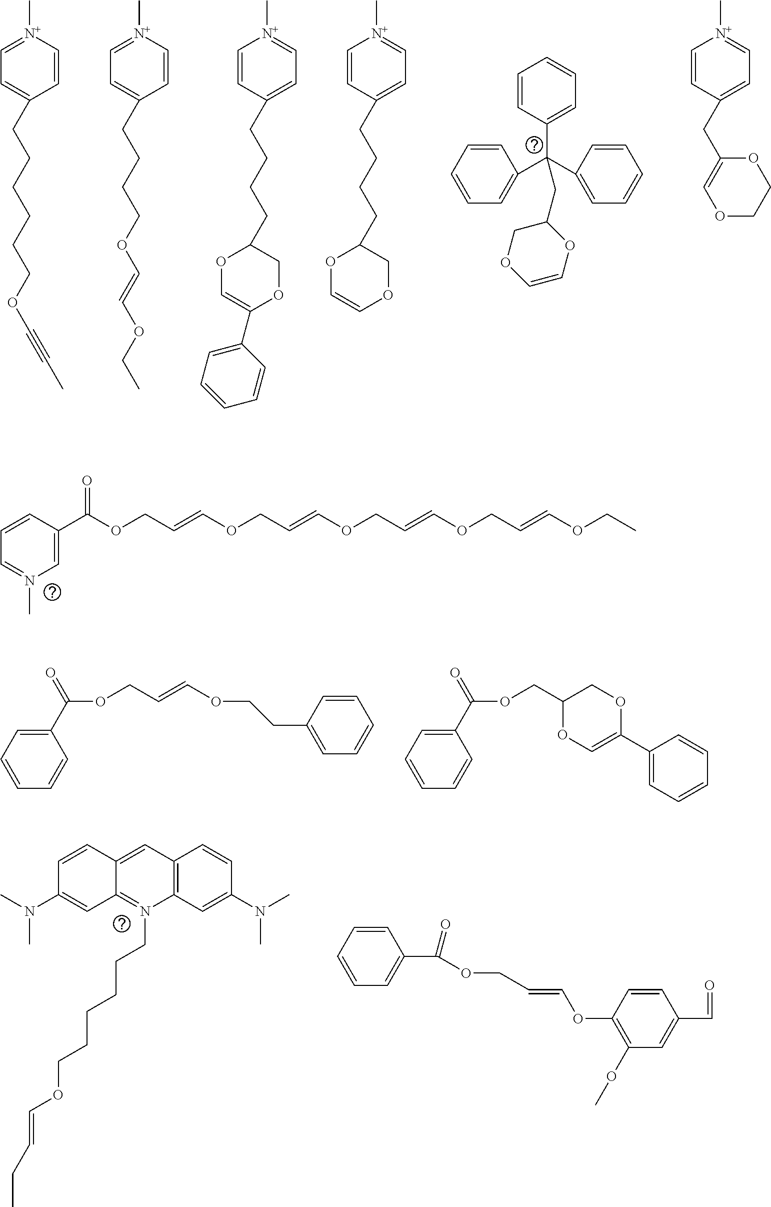

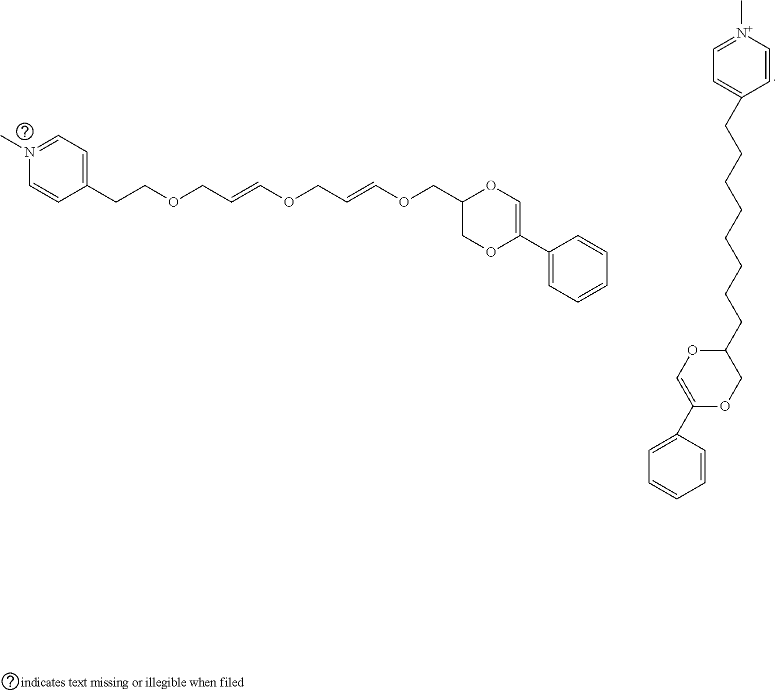

3. The compound of claim 1, wherein the compound forms an active product upon reaction with the reactive lipid species.

4. The compound of claim 3, wherein the active product is a food ingredient, a specific binding agent, a biocide, a fragrance, a cosmetic, a dye, an antioxidant, a fertilizer, a nutrient, a metabolite or a pharmaceutical.

5. (canceled)

6. The compound of claim 1, wherein the non-toxic or active product comprises an α-hydroxy aldehyde.

7. The compound of claim 6, wherein the α-hydroxy aldehyde is a sugar.

8. The compound of claim 3, wherein the active product inhibits or enhances the opening of a mitochondrial permeability transition pore (MPTP).

9. The compound of claim 8, wherein the non-toxic or active product comprises an α-dicarbonyl.

10. (canceled)

11. The compound of claim 1, wherein the reactive lipid species is in a mitochondrion.

12. The compound of claim 11, wherein the reactive lipid species is a cardiolipin hydroperoxide or a cardiolipin peroxyl radical.

13. The compound of claim 12, wherein the hydroperoxide or peroxyl radical is reduced to a (—O—) radical or non-radical moiety upon reaction with the compound.

14. The compound of claim 1, wherein the non-toxic or active product has a neutral or negative formal charge where the compound had a cationic charge.

15. The compound of claim 1, created upon reaction of a precursor compound with an enzyme.

16. The compound of claim 1, wherein the compound comprises a lipophilic moiety and a cationic moiety, and/or a lipophilic moiety and a water soluble moiety, and/or a lipophilic moiety and enzyme-activated cationic moiety.

17. (canceled)

18. The compound of claim 1, formulated in a pharmaceutical excipient.

19. The compound of claim 18, wherein the excipient comprises a liposome or a liposome-like vesicle.

20. (canceled)

21. The compound of claim 1, comprising a dienol ether, a divinyl thioether, a dienamine, an enol ether, a vinyl thioether, an enamine, or a combination thereof.

22. A method of reducing a reactive lipid species, the method comprising contacting the reactive lipid species with the compound of claim 1.

23. A method of treating a mitochondrion in a patient comprising a mitochondrial dysfunction, the method comprising contacting the mitochondrion with the compound of claim 1.

24. The method of claim 23, wherein the product reduces cardiolipin hydroperoxide or cardiolipin peroxyl radical to a (—O—) radical or non-radical moiety.

25-57. (canceled)
